(19) United States
(12) Patent Application Publication
(10) Pub. No.: US 2011/0183394 A1
(43) Pub. Date: Jul. 28, 2011

(54) METHOD OF PRODUCING YEAST BIOMASS

(75) Inventors: Philip John Livingstone Bell, New South Wales (AU); Paul Victor Attfield, New South Wales (AU); Arthur Kollaras, New South Wales (AU)

(73) Assignee: MICROBIOGEN PTY LTDC C/O Peter Hunt & Associates, Sydney, New South Wales (AU)

(21) Appl. No.: 13/001,329

(22) PCT Filed: Apr. 28, 2009
(86) PCT No.: PCT/AU09/00527

§ 371 Date: Mar. 24, 2011

(30) Foreign Application Priority Data
Jun. 27, 2008 (AU) 2008903291

(51) Int. Cl. C12P 7/06; C12P 1/02; C12N 1/16

(52) U.S. Cl. 435/161; 435/171; 435/255.2

(57) ABSTRACT

The invention relates to use of a substrate comprising C5 compound-containing material, in the growth of Saccharomyces yeast or the production of a product of Saccharomyces yeast, wherein the C5 compound-containing material is: (a) C5 compound-containing material obtained from lignocellulosic hydrolysate; (b) C5 compound-containing material obtained from fermentation of lignocellulosic hydrolysate; or (c) a mixture of (a) and (b). The invention also relates to a method of producing Saccharomyces yeast biomass or a product of Saccharomyces yeast using the substrate, and to methods of producing ethanol comprising incubating Saccharomyces yeast produced by the use or method. The invention further relates to strains of Saccharomyces yeast suitable for the use or methods.

### FIELD

The invention relates to a method of producing Saccharomyces yeast biomass or a product of a Saccharomyces yeast, substrates for growth of such yeast, to strains of Saccharomyces yeast, and use of the strains of Saccharomyces yeast in the production of yeast biomass, and yeast products such as ethanol. The invention has particular application for producing ethanol using Saccharomyces yeast and the invention is herein described in that context.

### BACKGROUND

Ethanol is an increasingly important renewable fuel for transport, as well as being important as an industrial chemical and 2-carbon chemicals precursor. Ethanol is mostly produced via fermentation of hexose based sugars by Saccharomyces yeast. The hexose based sugars are typically obtained from crop plants in the form of hydrolyzed starches from corn, wheat, barley, sorghum, millet, cassava, sweet potato, rice etc., or from sugar cane and sugar beet. The hexose based sugars that are fermented are typically glucose, fructose, sucrose and maltose.

Reliance on glucose, fructose, sucrose and maltose sugars of crop plants for production of ethanol and yeast biomass places pressure on supply of human and animal food sources. There is therefore an increasing demand to improve processes for the manufacture of ethanol and the production of yeast biomass. In particular, there is a need for increasing the efficiency by which plant based material is utilized for the production of yeast biomass and ethanol.

### SUMMARY

The inventors have found that Saccharomyces yeast biomass and Saccharomyces yeast products can be produced using substrates comprising C5 compound-containing material that is obtained from lignocellulosic hydrolysate and/or fermentation of lignocellulosic hydrolysate. Prior to the present invention, such material was considered to be unsuitable for production of Saccharomyces yeast biomass and Saccharomyces yeast products due to the presence of substances which inhibit the growth of Saccharomyces, and to a high concentration of C5 compounds which many Saccharomyces strains are unable to use for growth. In particular, C5 compound-containing material obtained from fermentation of lignocellulosic hydrolysate was considered to be a waste stream unsuitable for growth of Saccharomyces.

A first aspect provides use of a substrate comprising a C5 compound-containing material, in the growth of Saccharomyces yeast or the production of a product of Saccharomyces yeast wherein the C5 compound-containing material is:
(a) C5 compound-containing material obtained from lignocellulosic hydrolysate;
(b) C5 compound-containing material obtained from fermentation of lignocellulosic hydrolysate; or
(c) a mixture of (a) and (b).

A second aspect provides a method of producing Saccharomyces yeast biomass or a product of a Saccharomyces yeast, comprising incubating a substrate comprising a C5 compound-containing material with a Saccharomyces yeast in conditions which cause growth of the Saccharomyces yeast or production of the product, wherein the C5 compound-containing material is:

(a) C5 compound-containing material obtained from lignocellulosic hydrolysate;
(b) C5 compound-containing material obtained from fermentation of lignocellulosic hydrolysate; or
(c) a mixture of (a) and (b).

In one embodiment, the C5 compound-containing material is obtained from fermentation of lignocellulosic hydrolysate.

In another embodiment, the C5 compound-containing material is obtained from lignocellulosic hydrolysate.

In another embodiment, the C5 compound-containing material is a mixture of C5 compound-containing material obtained from lignocellulosic hydrolysate and C5 compound-containing material obtained from fermentation of lignocellulosic hydrolysate.

As used herein, “lignocellulosic hydrolysate” refers to material produced from lignocellulose in which the hemicellulose in the lignocellulose has undergone at least partial hydrolysis to release monomeric sugars from the hemicellulose polymer. Typically, cellulose in the lignocellulose is also at least partially hydrolysed to release sugar from the cellulose polymer.

Lignocellulose contains a rich source of sugars in the form of cellulose and hemicellulose. Cellulose is a polymer of linked glucose residues. Hemicellulose is a polymer which usually contains glucose, xylose, mannose, galactose and arabinose and other residues. However, the sugars in lignocellulose are unavailable to ethanologen organisms such as the yeast Saccharomyces cerevisiae, while contained in the polymers. The lignocellulose must therefore be processed prior to use in fermentation. Lignocellulose is processed using hydrolysis methods which release the sugar monomers present in the cellulose and hemicellulose to form a lignocellulosic hydrolysate. Processing of cellulose and hemicellulose polymers in lignocellulose (such as plant material) typically releases glucose, mannose, galactose, xylose and arabinose. Of these, glucose, mannose and galactose are hexose sugars while xylose and arabinose are pentose sugars. The released sugar residues in the hydrolysate are available for use in fermentation processes.

In addition to sugars, the lignocellulosic hydrolysate can contain a wide range of inhibitory chemicals such as is acetic acid, furans, phenolics and lignin breakdown products.

Fermentation of lignocellulosic hydrolysate using industrial ethanologens results in utilization of mostly C6 sugars present in the hydrolysate. As a consequence, the material remaining following fermentation of lignocellulosic hydrolysate is typically rich in C5 compounds such as xylose, as well as compounds such as glycerol, acetic acid, ethanol and other compounds which have carried through the fermentation. Furthermore, fermentation often results in a build-up of metabolites such as glycerol, acetic acid and ethanol, as well as other compounds that have carried through from the hydrolysate during the fermentation process. Such metabolites and compounds that have carried through from the hydrolysate can be inhibitory to the growth of Saccharomyces.

Thus, fermentation of lignocellulosic hydrolysates results in material which, prior to the present invention, was considered to form waste streams which were unsuitable for growth of Saccharomyces yeast. Such material can be of high biological and/or chemical oxygen demand and its disposal poses significant challenges.

In one form, the Saccharomyces yeast is capable of growth using xylose as a carbon source for growth.

Typically, the Saccharomyces yeast is capable of growth using xylose as a carbon source on a substrate obtained from fermentation of lignocellulosic hydrolysate.

By employing strains of Saccharomyces yeast which are capable of utilizing xylose, and typically one or more other carbon compounds, the inventors have found that what was previously considered to be a waste product could be used as a substrate for growth of Saccharomyces yeast biomass and/or production of Saccharomyces yeast products. The ability of the Saccharomyces yeast to grow on xylose, as well as other carbon compounds, found in the substrate reduces the biological oxygen demand and hence the chemical oxygen demand of the substrate.

As used herein, “Saccharomyces yeast biomass” is yeast cells of the genus Saccharomyces. As used herein, “production of Saccharomyces yeast biomass” is the growth of Saccharomyces yeast.

As used herein, the term “substrate” refers to a material used as a medium for growth of an organism and/or production of a product of an organism. The substrate contains a carbon source for the organism, and may contain a nitrogen source.

As used herein, the expression “C5 compound-containing material” is material containing one or more C5 carbon compounds. In one form, the C5 compound-containing material is xylose-containing material. Typically, the C5 compound-containing material comprises xylose, and may contain other C5 compounds. A C5 compound is a compound having 5 carbon atoms. Xylose is a C5 compound. Examples of other C5 compounds include arabinose, ribose and xylitol. Typically, the substrate contains a plurality of C5 compounds including xylose and one or more of xylitol, arabinose and ribose.

As used herein, “C5 compound-containing material obtained from fermentation of lignocellulosic hydrolysate” refers to C5 compound containing material present following fermentation of lignocellulosic hydrolysate, or a C5 compound-containing extract thereof.

As used herein, “C5 compound-containing material obtained from lignocellulosic hydrolysate” refers to C5 compound containing material present in lignocellulosic hydrolysate, or a C5 compound-containing extract thereof.

The substrate comprises a sufficient amount of the C5 compound-containing material to support growth of the Saccharomyces yeast.

In one form, the substrate comprises at least 20%, 30%, 40%, 50%, 60%, 65%, 70%, 75%, 80%, 85%, 90%, 95%, 99% or 100% C5 compound-containing material obtained from fermentation of lignocellulosic hydrolysate.

In one form, the C5 compound-containing material is dunder. As used herein, the term “dunder” refers to the liquid remaining following fermentation of lignocellulosic hydrolysate and subsequent extraction of the ethanol. The amount of dunder in the substrate is typically the amount sufficient to provide a carbon source for growth of the yeast. For example, the substrate may comprise at least 20%, 30%, 40%, 50%, 60%, 65%, 70%, 75%, 80%, 85%, 90%, 95% or 99%, dunder. In one form, the substrate is dunder.

In another form, the C5 compound-containing material is the liquid and solids obtained from fermentation of lignocellulosic hydrolysate. In this form, following fermentation of the lignocellulosic hydrolysate and extraction of alcohol, the material remaining may be used to form the substrate without further removal of solid matter.

In one form, the substrate comprises at least 20%, 30%, 40%, 50%, 60%, 65%, 70%, 75%, 80%, 85%, 90%, 95%, 99% or 100% C5 compound-containing material obtained from lignocellulosic hydrolysate.

In one form, the C5 compound-containing material is lignocellulosic hydrolysate.

In one form, the C5 compound-containing material is a mixture of lignocellulosic hydrolysate and dunder. The lignocellulosic hydrolysate and dunder can be mixed in any ratio. Examples of suitable ratios of dunder lignocellulosic hydrolysate include: 1:1, 1:1.25, 1:1.5, 1:1.75, 1:2, 1:3, 1:4, 1:5, 1:6, 1:7, 1:8, 1:9, 1:10, 1.25:1, 1.5:1, 1.75:1, 2:1, 3:1, 4:1, 5:1, 6:1, 7:1, 8:1, 9:1, 10:1.

In one embodiment, the substrate is the C5 compound-containing material.

As used herein, a Saccharomyces yeast is a yeast of the genus Saccharomyces as defined in Kurtzman et al. (2003) FEMS Yeast Research 4:235-245.

In one embodiment, the substrate is utilized by growing the Saccharomyces yeast on the substrate. Typically, the Saccharomyces yeast is grown aerobically on the substrate. In embodiments where the substrate comprises C5 compound-containing material obtained from fermentation of ligocellulosic hydrolysate, aerobic growth of the Saccharomyces yeast on the substrate allows carbon sources that were not converted into ethanol or extracted from the fermentation process to be effectively removed from the waste stream and converted into useful yeast biomass. This has the added advantage of reducing the chemical and biological oxygen demand of the substrate.

A large quantity of the Saccharomyces yeast cells can be produced from the xylose and other carbon sources in the substrate. The Saccharomyces yeast can then be used for any application for which the Saccharomyces yeast are suitable. As described herein, the Saccharomyces yeast can be used for inoculation of lignocellulosic hydrolysate for fermentation of the lignocellulosic hydrolysate, or for inoculation of substrate for production of further yeast biomass or yeast product. The Saccharomyces yeast can also be used as inocula for traditional fermentations such as baking, brewing, wine, potable or non-potable distilled spirits etc., or as animal feed stock, or for Saccharomyces yeast products.

In one form, the lignocellulosic hydrolysate is a pH adjusted lignocellulosic hydrolysate. As used herein, a “pH adjusted lignocellulosic hydrolysate” is a lignocellulosic hydrolysate which has a pH which supports growth of, and/or fermentation by, Saccharomyces yeast. The pH adjusted hydrolysate may comprise an acid hydrolyzed lignocellulosic hydrolysate and an alkaline agent. In one form, the alkaline agent is ammonia.

The Saccharomyces yeast grown on the substrate may be used for fermentation of lignocellulosic hydrolysate. Thus, a third aspect provides a method of producing ethanol comprising the steps of:
(a) incubating a Saccharomyces yeast produced by the use of the first aspect or method of the second aspect and a pH adjusted lignocellulosic hydrolysate under conditions which cause fermentation of the lignocellulosic hydrolysate to produce ethanol and a C5 compound-containing material; and
(b) isolating the ethanol.

The method of the third aspect may comprise the further step of:
(c) growing Saccharomyces yeast on substrate comprising:
   (i) the C5 compound-containing material produced in step (a);
   (ii) C5 compound-containing material obtained from lignocellulosic hydrolysate; or
   (iii) a mixture of (i) and (ii),
      wherein the Saccharomyces yeast is capable of utilizing xylose as a carbon source for growth on the substrate.

The method of the third aspect may comprise the further step of:
(d) repeating steps (a) to (c) using the Saccharomyces yeast grown in step (c).

A fourth aspect provides a method of producing ethanol and Saccharomyces yeast, comprising the steps of:
(a) incubating a pH adjusted lignocellulosic hydrolysate, and a Saccharomyces yeast capable of utilizing xylose as a carbon source for growth on a substrate obtained from fermentation of lignocellulosic hydrolysate, under conditions which cause fermentation of the lignocellulosic hydrolysate to produce ethanol and a C5 compound-containing material;
(b) isolating the ethanol; and
(c) growing Saccharomyces yeast on substrate comprising:
   (i) the C5 compound-containing material produced in step (a);
   (ii) C5 compound-containing material obtained from lignocellulosic hydrolysate; or
   (iii) a mixture of (i) and (ii),
      wherein the Saccharomyces yeast is capable of utilizing xylose as a carbon source for growth on the substrate.

The method of the fourth aspect may further comprise the step of:
(d) repeating steps (a) to (c) using the Saccharomyces yeast grown in step (c).

A fifth aspect provides a method of producing ethanol, comprising the steps of:
(a) providing Saccharomyces yeast capable of utilizing xylose as a carbon source for growth on a substrate obtained from fermentation of lignocellulosic hydrolysate, and a lignocellulosic hydrolysate;
(b) pH adjusting the lignocellulosic hydrolysate for fermentation by the Saccharomyces yeast, the pH adjusting comprising adding ammonia to adjust the pH of the hydrolysate to a pH which supports fermentation by the Saccharomyces yeast;
(c) incubating the Saccharomyces yeast and pH adjusted lignocellulosic hydrolysate in conditions which cause fermentation of the lignocellulosic hydrolysate to produce ethanol and a C5 compound-containing material;
(d) isolating the ethanol.

A sixth aspect provides a method of producing ethanol and Saccharomyces yeast biomass, comprising the steps of:
(a) providing Saccharomyces yeast capable of utilizing am xylose as a carbon source for growth on a substrate obtained from fermentation of lignocellulosic hydrolysate, and a lignocellulosic hydrolysate;
(b) pH adjusting the lignocellulosic hydrolysate for fermentation by the Saccharomyces yeast, the pH adjusting comprising adding ammonia to adjust the pH of the hydrolysate to a pH which supports fermentation by the Saccharomyces yeast;
(c) incubating the Saccharomyces yeast and pH adjusted lignocellulosic hydrolysate in conditions which cause fermentation of the lignocellulosic hydrolysate to produce ethanol and a C5 compound-containing material;
(d) isolating the ethanol; and
(e) growing Saccharomyces yeast on substrate comprising:
   (i) the C5 compound-containing material produced in step (c);
   (ii) C5 compound-containing material obtained from lignocellulosic hydrolysate; or
   (iii) a mixture of (i) and (ii),
      wherein the Saccharomyces yeast is capable of utilizing xylose as a carbon source for growth on the substrate.

The method of the sixth aspect may comprise the further step of:
(f) repeating steps (a) to (e) using the Saccharomyces yeast grown in step (e).

A seventh aspect provides a substrate comprising C5 compound-containing material that is:

(a) C5 compound-containing material obtained from lignocellulosic hydrolysate;
(b) C5 compound-containing material obtained from fermentation of a lignocellulosic hydrolysate; or
(c) a mixture of (a) and (b),
for use in the production of Saccharomyces yeast or a product of a Saccharomyces yeast, the Saccharomyces yeast being capable of utilizing xylose as a carbon source for growth on the substrate.

The Saccharomyces yeast may be recombinant or non-recombinant. In a particular form, the Saccharomyces yeast is non-recombinant.

The Saccharomyces yeast is typically a Saccharomyces strain which produces at least a 10-fold increase in biomass in Test T1.

In one form, the Saccharomyces yeast is a strain which is capable of growing aerobically in pH adjusted lignocellulosic hydrolysate.

In one form, the Saccharomyces yeast is a strain of Saccharomyces cerevisiae.

The Saccharomyces yeast are typically prototrophic and are therefore capable of growing on substrates without the need for additions of complex nutrients such as amino acids, nucleic acid bases, yeast extracts, malt extracts, peptone or other complex and expensive supplements that might be used to assist growth and fermentation by yeasts. The Saccharomyces yeast therefore only requires that sufficient nitrogen be added to lignocellulosic hydrolysates and has the characteristic of being able to grow using industrially economic substrate additions. Inexpensive substrate additions might include for example, ammonia, ammonium salts, urea, mono- or di-ammonium phosphate or other industrially economic sources of nitrogen and/or phosphate.

In a particular form, the Saccharomyces yeast is selected from the group consisting of a strain of Saccharomyces cerevisiae having NMI accession no. V08/013,411, or a mutant or derivative thereof which has the defining properties of NMI V08/013,411, and a strain of Saccharomyces cerevisiae having NMI accession no. V09/005,064, or a mutant or derivative thereof which has the defining properties of NMI V09/005,064.

Strain V08/013,411 and strain V09/005,064 are capable of tolerating high levels of inhibitors in lignocellulosic hydrolysate and in the substrate. Strains V08/013,411 and V09/005,064 are also capable of growth in the substrate using xylose as a sole carbon source for growth.

An eighth aspect provides a Saccharomyces yeast selected from the group consisting of: a strain having NMI accession no. V08/013,411, or a mutant or derivative thereof which has the defining properties of NMI accession no. V08/013,411; and a strain having NMI accession no. V09/005,064, or a mutant or derivative thereof which has the defining properties of NMI accession no. V09/005,064.

A ninth aspect provides a method of producing ethanol, comprising the steps of:
(a) incubating a Saccharomyces yeast according to the eighth aspect and pH adjusted lignocellulosic hydrolysate in conditions which cause fermentation of the lignocellulosic hydrolysate to produce ethanol and a C5 compound-containing material; and
(b) isolating the ethanol.

The method of the ninth aspect may comprise the further step of:
(c) growing Saccharomyces yeast of the eighth aspect on substrate comprising:
   (i) the C5 compound-containing material produced in step (a);
   (ii) C5 compound-containing material obtained from lignocellulosic hydrolysate; or
   (iii) a mixture of (i) and (ii).

The method of the ninth aspect may comprise the further step of:
(d) repeating steps (a) to (c) using the Saccharomyces yeast grown in step (c).

A tenth aspect provides a method of producing ethanol and Saccharomyces yeast, comprising the steps of:
(a) incubating a Saccharomyces yeast strain according to the eighth aspect and a pH adjusted lignocellulosic hydrolysate under conditions which cause fermentation of the lignocellulosic hydrolysate to produce ethanol and a C5 compound-containing material;
(b) isolating the ethanol; and
(c) growing Saccharomyces yeast of the eighth aspect on substrate comprising:
   (i) the C5 compound-containing material produced in step (a);
   (ii) C5 compound-containing material obtained from lignocellulosic hydrolysate; or
   (iii) a mixture of (i) and (ii).

The method of the tenth aspect may further comprise the step of:
(d) repeating steps (a) to (c) using the Saccharomyces yeast grown in step (c).

In one embodiment, the substrate is the C5 compound-containing material. In one form, the substrate is dunder.

The pH adjusted lignocellulosic hydrolysate typically has a pH which supports fermentation by the Saccharomyces yeast.

In one embodiment, the pH adjusted lignocellulosic hydrolysate comprises a lignocellulosic hydrolysate and an alkaline agent. In one form, the alkaline agent is ammonia.

In embodiments in which the Saccharomyces yeast is used for fermentation of pH adjusted lignocellulosic hydrolysate, the Saccharomyces yeast are incubated with the pH adjusted lignocellulosic hydrolysate at a density which is sufficient to permit fermentation of the hydrolysate. The Saccharomyces yeast are typically incubated with the pH adjusted lignocellulosic hydrolysate at a density of: at least about 2×10⁶; at least about 2×10⁷; at least about 2×10⁸; at least about 2×10⁹, cells per milliliter of hydrolysate.

In embodiments in which the Saccharomyces yeast are grown on the substrate, the substrate is incubated with Saccharomyces yeast at a density which is sufficient to permit growth of the Saccharomyces yeast. Typically, the Saccharomyces yeast are incubated with the substrate at a density of: at least about 2×10⁶, at least about 2×10⁷; at least about 2×10⁸; at least about 2×10⁹, cells per millilitre of hydrolysate.

In embodiments in which the Saccharomyces yeast are grown on the substrate, the Saccharomyces yeast are typically grown aerobically on the substrate.

An eleventh aspect provides a method of pH adjusting a lignocellulosic hydrolysate by adding ammonia to a lignocellulosic hydrolysate to adjust the pH to a pH which supports fermentation by a Saccharomyces yeast.

A twelfth aspect provides a pH adjusted lignocellulosic hydrolysate suitable for fermentation by a Saccharomyces yeast comprising a lignocellulosic hydrolysate and ammonia.

The use of ammonia serves to adjust the pH of the lignocellulosic hydrolysate to a range suitable to support yeast growth and fermentation. In addition, ammonia provides a readily available, inexpensive nitrogen source for the yeast to utilize.

The inventors have found that pH adjusting of the lignocellulosic hydrolysate can be achieved using ammonia rather than using compounds such as calcium hydroxide, calcium carbonate or sodium hydroxide. This can be advantageous because, for example, calcium salts can generate high levels of insoluble ash such as gypsum. High levels of ash produced during the production of pH adjusted lignocellulosic hydrolyzates poses disposal challenges.

Also, high levels of calcium salts can cause downstream processing problems due to the ability of calcium salts to produce scale within boilers. In addition, avoidance of the use of calcium hydroxide, calcium carbonate or sodium hydroxide for pH adjusting the hydrolysate avoids the associated ionic inhibitory and osmotic stress produced by these ions. Further, by using ammonia in the pH adjusting of the hydrolysate, nitrogen is provided for Saccharomyces yeast propagation and fermentation.

Overliming, caused by the use of calcium hydroxide as a pH adjusting agent, has been widely used in the neutralisation of lignocellulose because of its utility in reducing the inhibitory characteristics of the hydrolysate, thus allowing yeast to ferment the hydrolysate without further purification. The avoidance of overliming provides hydrolysates that are more inhibitory than would be the case if calcium agents were used for pH adjusting purposes. Thus, in one form, Saccharomyces yeast that are resistant to ammonia neutralised hydrolysate are used in this process. Examples of such Saccharomyces yeast are strains V08/013,411 and 09/005,064.

Yeast as described herein may be grown on carbon sources present in lignocellulosic hydrolysate, to provide biomass for subsequent fermentation of lignocellulosic hydrolysate, or for the production of excess yeast for sale or use in traditional fermentations such as baking, brewing, wine, potable and non-potable distilled spirits etc., or as additives, food, protein supplements, extracts etc.

A thirteenth aspect provides a method of producing a Saccharomyces yeast biomass or a product of a Saccharomyces yeast, comprising incubating a lignocellulosic hydrolysate with a Saccharomyces yeast under conditions which cause growth of the Saccharomyces yeast or production of the product, wherein the Saccharomyces yeast is capable of utilizing xylose as a carbon source for growth on a substrate obtained from fermentation of lignocellulosic hydrolysate.

In one embodiment, the Saccharomyces yeast is a Saccharomyces yeast of the eighth aspect.

A fourteenth aspect provides a method of producing Saccharomyces yeast biomass or a product of Saccharomyces yeast, comprising incubating a substrate comprising C5 compound-containing material with a Saccharomyces yeast of the eighth aspect under conditions which cause growth of the Saccharomyces yeast or production of the product, wherein the C5 compound-containing material is:
(a) C5 compound-containing material obtained from lignocellulosic hydrolysate;
(b) C5 compound-containing material obtained from fermentation of lignocellulosic hydrolysate; or
(c) a mixture of (a) and (b).

Typically, the lignocellulosic hydrolysate is pH adjusted. In one form, the pH adjusted hydrolysate comprises lignocellulosic hydrolysate and ammonia.

Typically, the yeast is grown on the substrate. The yeast is typically grown aerobically on the substrate.

A fifteenth aspect provides a method of reducing the biological oxygen demand of a substrate comprising C5 compound-containing material obtained from fermentation of lignocellulosic hydrolysate, comprising incubating a Saccharomyces yeast with the substrate under conditions which cause growth of the Saccharomyces yeast.

In one embodiment, the Saccharomyces yeast is Saccharomyces yeast of the eighth aspect.

### BRIEF DESCRIPTION OF THE DRAWINGS

An embodiment of the invention will be described with reference to the accompanying drawings. The particularity of the drawings and its related description is to be understood as not superseding the generality of the preceding broad description of the invention.

In the drawings:

FIG. 1 shows a schematic diagram of an embodiment of the method for producing ethanol showing inputs and outputs at stages of the process; and

FIG. 2 is a block diagram of the ethanol production process of FIG. 1.

### DETAILED DESCRIPTION

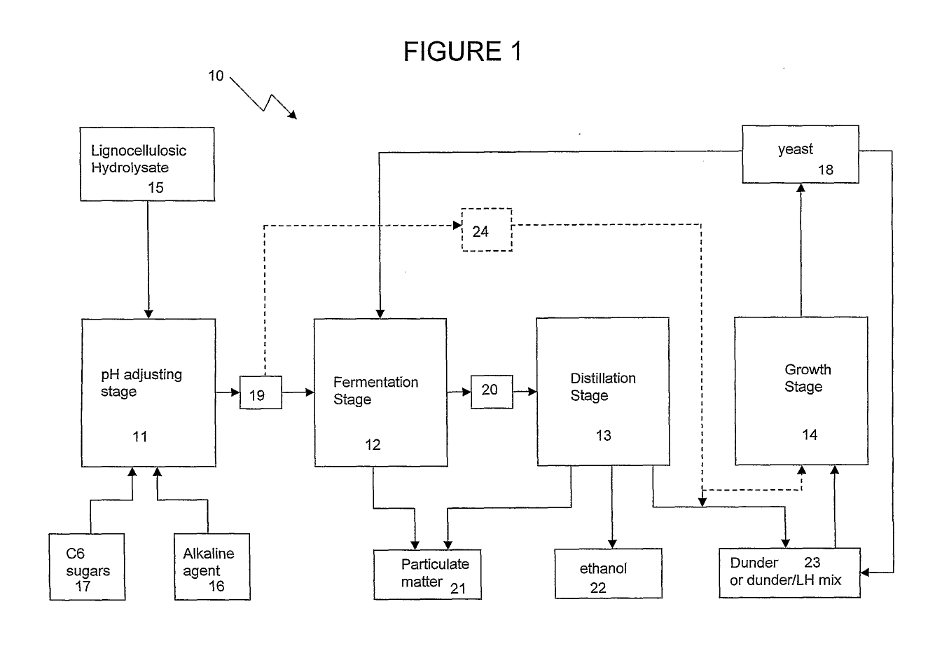

FIG. 1 illustrates a general overview of an ethanol production process (10) according to one embodiment of the invention. FIG. 1 illustrates the main stages of the process (10) and the inputs and outputs in the process. The process may be divided into 4 main stages, being the pH adjusting stage (11), the fermentation stage (12), the distillation stage (13) and the growth stage (14).

In the pH adjusting stage, the lignocellulosic hydrolysate is pH adjusted. Lignocellulosic hydrolysate is produced by hydrolysis of lignocellulose to release sugars which are present in the cellulose and hemicellulose polymers which make up lignocellulose. A lignocellulosic hydrolysate contains carbon compounds such as glucose, mannose, galactose, xylose and arabinose as well as compounds that can be inhibitory to Saccharomyces yeast such as organic acids, furans (e.g., furfural, furfuryl alcohol and 5-hydroxymethylfurfural) and lignophenolic compounds. The lignocellulosic hydrolysate may be produced on site, or obtained from an offsite source. In one form, the pH adjusting comprises the addition of an alkaline agent to adjust the pH of the hydrolysate to a pH suitable for the fermentation by a Saccharomyces yeast. The alkaline agent may be calcium hydroxide, calcium carbonate, sodium hydroxide, ammonia, or combinations thereof. In the particular form illustrated, the alkaline agent is ammonia. Thus, the main input for the pH adjusting stage is lignocellulosic hydrolysate (15) and an alkaline agent in the form of ammonia (16). In addition, C6 sugars (17) in the form of, for example, C6 sugars released via cellulose hydrolysis, molasses, cane juice, sugar syrups from hydrolysed starches, maltodextrins, raw sugar juice, glucose, galactose, sucrose or other C6 compounds, or combinations thereof, may also be input for the pH adjusting stage. The output from the pH adjusting stage is pH adjusted lignocellulosic hydrolysate (19). The pH of the pH adjusted lignocellulosic hydrolysate is typically from 2.5 to 7. Typically the pH is a pH from: 2.5 to 6.5; 2.5 to 6.0; 2.5 to 5.5; 2.5 to 5.0; 3.0 to 5.0; 3.5 to 5.0; 4.0 to 5.5.

In the fermentation stage, Saccharomyces yeast (18) and the pH adjusted hydrolysate (19) are incubated under conditions which permit fermentation of C6 sugars in the hydrolysate. C6 substrates can also be provided in the fermentation stage in the form of, for example, cellulosic polymers, starches or dextrins. Enzymes with celluloytic activities, starch or dextrin hydrolyzing activities can be added into the fermentation stage to permit simultaneous saccharification and yeast fermentation of the resultant free fermentable sugars. The Saccharomyces yeast (18) may be any Saccharomyces yeast which is capable of fermentation of at least one C6 sugar in pH adjusted lignocellulosic hydrolysate, and growth on a substrate obtained from fermentation of lignocellulosic hydrolysate.

Saccharomyces yeast strains that are suitable for use in the fermentation are Saccharomyces yeast strains which are capable of fermenting C6 sugars and utilising xylose as a carbon source for growth, and which are capable of growth in the presence of inhibitors found in dunder. Suitable Saccharomyces yeast strains include Saccharomyces yeast strains having the ability to grow aerobically on xylose and any one or more of acetate, glycerol, ethanol, glucose, fructose, mannose and galactose, typically in the presence of inhibitors at concentrations found in dunder. In one form, the Saccharomyces yeast is one or more strains of Saccharomyces yeast from the genus Saccharomyces which is capable of utilizing xylose as a carbon source for growth. Typically, the Saccharomyces yeast is one or more strains of Saccharomyces cerevisiae. The strain of Saccharomyces cerevisiae may be, for example, NMI accession no. V08/013,411, or a mutant or derivative of NMI V08/013,411 which has the defining properties of NMI V08/013,411, or NMI accession no. V09/005,064, or a mutant or derivative of NMI V09/005,064 which has the defining properties of NMI V09/005,064. Such Saccharomyces cerevisiae strains have the ability to grow aerobically on a diverse range of carbon sources including xylose, glycerol, acetic acid, xylitol. Such strains are also able to immediately start rapid fermentation of glucose, fructose or mannose present in a lignocellulosic hydrolysate provided a sufficient inoculum level is used. In a particular form, the Saccharomyces yeast is strain V09/005,064.

In the initial cycle of the process, Saccharomyces yeast is typically provided by growing a stock culture of the Saccharomyces yeast aerobically on a substrate, typically dunder, obtained from fermentation of a lignocellulosic hydrolysate, or on pH adjusted lignocellulosic hydrolysate, or on a mixture of dunder and pH adjusted lignocellulosic hydrolysate. For subsequent cycles of the process, Saccharomyces yeast grown on dunder in the growth stage may be input into the fermentation stage. Growth of Saccharomyces yeast on the dunder permits the production of high levels of Saccharomyces yeast that are preconditioned to be resistant to the inhibitors present in lignocellulosic hydrolysates. The Saccharomyces yeast produced on the dunder is therefore suitable for efficient growth and fermentation in lignocellulosic hydrolysates. Thus, the growth of Saccharomyces yeast on substrate comprising C5 compound-containing material obtained from fermentation of lignocellulosic hydrolysate provides a pre-adapted inoculum suitable for efficient fermentation of lignocellulosic hydrolysates, whilst also reducing the biological oxygen demand and chemical oxygen demand of the dunder. During the fermentation stage, acid is produced by the yeast as a metabolic by-product of the fermentation. Bases or buffering agents may therefore be input at the fermentation stage as required to maintain the fermenting lignocellulosic hydrolysate at a pH which permits the yeast to continue to ferment the lignocellulosic hydrolysate. Examples of suitable bases which can be used to maintain the pH of the fermenting lignocellulosic hydrolysate include ammonia, sodium hydroxide, potassium hydroxide, calcium hydroxide, or combinations thereof. Buffering of pH can also be achieved by addition of, for example, citric acid or citrate.

Following the fermentation stage, fermented hydrolysate (20) is typically output in the form of a material comprising ethanol, xylose and other C5 compounds, residual C6 sugars, glycerol, acetate, inhibitors from the hydrolysate and from Saccharomyces yeast metabolism and residual solids. The output from the fermentation stage serves as input for the distillation stage (13). Output from the fermentation stage is ethanol (22), particulate matter (21) in the form of lignins, Saccharomyces yeast, residual cellulose and hemicellulose, and xylans, which can be discarded or used for energy generation by, for example, burning, or through products of anaerobic digestion.

In the distillation stage (13), material from the fermentation stage is heated to distill the ethanol (22). Once the ethanol has been evaporated, the liquid that remains is dunder (23). Thus, output from the distillation stage is ethanol (22) dunder (23) and particulate matter (21). The dunder (23) is high in xylose and other carbon compounds, which serve as a carbon source for aerobic Saccharomyces yeast growth in the growth stage (14).

In the growth stage (14), Saccharomyces yeast is grown aerobically on the dunder (23). The dunder (23) and Saccharomyces yeast (18) are therefore input for the growth stage. The output of the growth stage is Saccharomyces yeast (18) and spent dunder. Because much of the xylose and other carbon compounds in the dunder are used up in the growth stage, the biological and chemical oxygen demand of the spent dunder is reduced, thereby reducing the environmental impact of disposal of the spent dunder. In the first cycle of the process, Saccharomyces yeast for the growth stage is provided from a stock culture. However, Saccharomyces yeast output from the growth stage may be used as input for both the growth stage and the fermentation stage in subsequent cycles of the process.

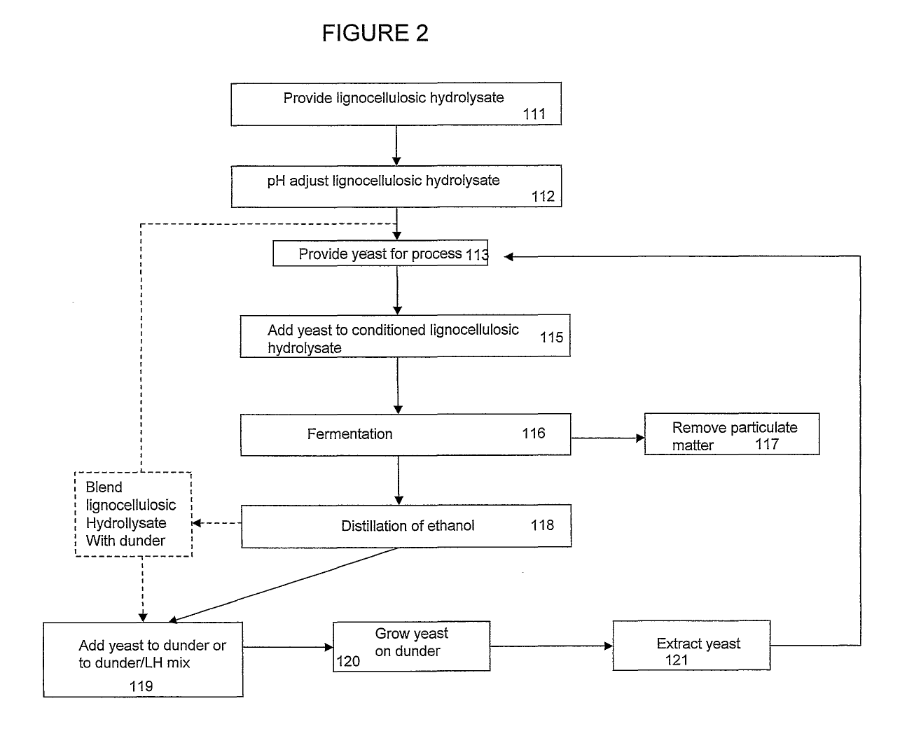

FIG. 2 illustrates the main process steps for the ethanol production process (10) described above. In a first step (111), the lignocellulosic hydrolysate (15) is provided. The lignocellulosic hydrolysate (15) may be provided from an offsite source, or by preparing the hydrolysate on site. Lignocellulosic hydrolysate may be prepared by hydrolyzing lignocellulosic biomass. The lignocellulosic biomass may be any biomass containing hemicellulose and/or cellulose. Typically, the biomass is plant biomass. Examples of lignocellulosic biomass include hardwood, softwood, corn stover, sugarcane bagasse, sugarcane trash, grass, algae or other plant matter, waste papers, recycled paper, waste streams of paper industries, wood molasses, municipal solid wastes and green wastes.

Methods for hydrolysis of lignocellulosic biomass are known in the art and include, for example, acid hydrolysis, enzymatic hydrolysis, ammonia fibre/freeze explosion (AFEX), ammonia recycled percolation (ARP), organosolv, etc. Methods for acid hydrolysis of lignocellulosic biomass are described in, for example, U.S. Pat. No. 4,612,286 and U.S. Pat. No. 6,063,204. Acidification may be brought about by using for example, intrinsic acidification of lignocellulosics during so-called autohydrolysis, addition of acidifying agents such as sulphuric acid, sulphur dioxide, nitric acid, hydrochloric acid, phosphoric acid, acetic acid, etc. Methods for AFEX are described in, for example, U.S. Pat. No. 4,600,590. Methods for ARP are described in, for example, Kim et al. (2003) Bioresource Technology, 90:39-47. Methods for enzymatic hydrolysis are described in, for example, U.S. Pat. No. 3,642,580. Methods for organosolv are described in, for example, U.S. Pat. No. 4,409,032. Solvents used in organosolv may include, for example, ethanol, acetone etc.

The pH of lignocellulosic hydrolysate is typically about 1, which is unsuitable for fermentation by Saccharomyces yeast such as Saccharomyces cerevisiae. Thus, in a second step (112), the lignocellulosic hydrolysate is pH adjusted prior to fermentation. The pH adjusting step comprises the addition of an alkaline agent such as ammonia (16). The ammonia is in one form added from a 32% concentrated ammonia solution. The ammonia is added until a pH of typically about 5.0 to 5.5 is achieved. Alternatively, the alkaline agent may be, for example, calcium hydroxide, calcium carbonate or sodium hydroxide. In addition to adjusting the pH, C6 sugars may be added to the pH adjusted hydrolysate (17) in the form of hydrolysed cellulose, molasses, cane juice, syrups from hydrolysed starches, maltodextrins to assist in the fermentation. Additional nitrogen and phosphorus may also be provided by the addition of for example, urea, mono- and/or di-ammonium phosphate. In addition, enzymes may be added at this stage to assist in further breakdown of cellulose or hemicellulose polymers. Suitable enzymes include cellulases, cellobiases, xylanases, glucosidases etc. Commercially available enzyme preparations for breakdown of cellulose or hemicellulose polymers include, for example, Spirizyme Fuel, Cellulclast and Novozym 188 (available from Novozymes Australia Pty Ltd).

In a third step (113), an initial inoculum of Saccharomyces yeast is provided, typically in the form of Saccharomyces strain NMI V08/013,411 or NMI V09/005,064. The Saccharomyces yeast is used to inoculate the pH adjusted hydrolysate in a fourth step (115). In one form, the Saccharomyces yeast is inoculated into the hydrolysate in a batch fermenter to a density of about 2×10⁶ to about 2×10⁹ cells per milliliter of hydrolysate. Subsequent cycles of the process may use Saccharomyces yeast grown on the dunder to inoculate the pH adjusted lignocellulosic hydrolysate. The use of Saccharomyces yeast at a high density of about 2×10⁷ to about 2×10⁹ cells per milliliter of lignocellulosic hydrolysate in one form detoxifies the hydrolysate. The use of high density inoculation of the hydrolysate with Saccharomyces yeast eliminates the need for detoxification using over-liming or other treatments.

In a fifth step (116), the pH adjusted lignocellulosic hydrolysate is fermented by the Saccharomyces yeast (18). The Saccharomyces yeast and hydrolysate are incubated at 30 degrees Celsius for typically about 1 to 4 days, more typically about 2 to 3 days, in a batch fermenter to allow fermentation of the hydrolysate to proceed. Fermentation of the lignocellulosic hydrolysate results in utilization of C6 sugars in the pH adjusted hydrolysate and production of ethanol.

Material from the fermentation is subjected to distillation (118) in a sixth step to extract the ethanol (22). Distillation is typically achieved by heating the material from the fermentation to evaporate the ethanol. The evaporated ethanol is collected following condensation.

Following distillation of the ethanol, the remaining material is separated into dunder and solids, and the dunder is used in a seventh step (119) as a substrate for growth of Saccharomyces yeast, such as Saccharomyces strain V08/013,411 or V09/005,064. Thus, the dunder (23) from the distillation is inoculated with the Saccharomyces yeast and the Saccharomyces yeast are grown aerobically by agitation of the Saccharomyces yeast and dunder at temperature of from 25-42° C., typically 30-37° C., more typically 35-37° C. (120). The Saccharomyces yeast are then extracted from the dunder by, for example, centrifugation or filtration (121). The Saccharomyces yeast resulting from growth on the dunder (122) is then used as inoculum for fresh pH adjusted hydrolysate in subsequent fermentations (116). In addition, Saccharomyces yeast (18) grown on the dunder is used to inoculate dunder obtained from the fermentation of hydrolysate to thereby grow further Saccharomyces yeast. Thus, with each cycle of the process, Saccharomyces yeast is produced by growth on the dunder and the resulting Saccharomyces yeast can be used for the next cycle of fermentation as well as inoculum for the dunder in preparation for the next cycle. The process therefore produces large amounts of Saccharomyces yeast biomass as well as ethanol.

An alternative embodiment is illustrated by the dashed line in FIGS. 1 and 2, in which the substrate (23) is a mixture of dunder and lignocellulosic hydrolysate. In this regard, a portion of pH adjusted lignocellulosic hydrolysate (24) is retained from the pH adjusting stage (11) for mixing with the dunder (23) at the growth stage. Following distillation of the ethanol, dunder (23) from the distillation is mixed with the lignocellulosic hydrolysate (24) and the mixture is used as a substrate for growth of the Saccharomyces yeast using the growth conditions as described above. Alternatively, the lignocellulosic hydrolysate (24) may be added to the yeast/dunder mix in the growth stage (14). The lignocellulosic hydrolysate and dunder can be mixed in any ratio. Examples of suitable ratios of dunder:lignocellulosic hydrolysate include: 1:1, 1:1.25, 1:1.5, 1:1.75, 1:2, 1:3, 1:4, 1:5, 1:6, 1:7, 1:8, 1:9, 1:10, 1.25:1, 1.5:1, 1.75:1, 2:1, 3:1, 4:1, 5:1, 6:1, 7:1, 8:1, 9:1, 10:1. Addition of the lignocellulosic hydrolysate to the dunder provides additional C5 compound-containing material for growth of the Saccharomyces yeast and is therefore useful when more biomass is required than can be produced on the dunder alone. The Saccharomyces yeast biomass produced may be used to inoculate the pH adjusted lignocellulosic hydrolysate at the fermentation stage, or for any other purpose suitable for Saccharomyces yeast.

The invention also relates to an isolated strain of Saccharomyces that is selected from the group consisting of NMI accession no. V08/013,411, or a mutant or derivative thereof which has the defining properties of NMI accession no. V08/013,411, and NMI accession no. NMI V09/005,064, or a mutant or derivative thereof which has the defining properties of V09/005,064.

The inventors have isolated and deposited under the Budapest Treaty at the National Measurement Institute Saccharomyces cerevisiae strains under deposit accession nos. V08/013,411 and V09/005,064. Deposit accession nos. V08/013,411 and V09/005,064 have the following general properties:
(a) ability to utilise xylose as a sole carbon source;
(b) ability to grow in the presence of inhibitors found in dunder;
(c) ability to sporulate and mate with Saccharomyces cerevisiae.

Deposit accession nos. NMI V08/013,411 and V09/005,064 have the following defining properties:
i. give at least a ten-fold increase in biomass in Test T1;
ii. sporulate, and produce progeny when the germinated spores are mated with germinated spores of a tester strain of Saccharomyces cerevisiae;
iii. grow in dunder produced from fermentation of lignocellulosic hydrolysate.

An example of a suitable tester strain of Saccharomyces cerevisiae is W303-1A. W303-1A is readily available from the Yeast Genetic Stock Center at the ATCC, USA. Methods for sporulating, germinating and mating Saccharomyces yeast are described below.

Saccharomyces strains V08/013,441 and V09/005,064 are capable of growth on xylose as a sole carbon source. Moreover, strains V08/013,441 and V09/005,064 are able to grow in the inhibitory conditions present in dunder. Accordingly, strains V08/013,441 and V09/005,064 can readily be grown in dunder. Furthermore strains V08/013,441 and V09/005,064 can ferment C6 sugars in the presence of lignocellulosic hydrolysate.

In one embodiment, the Saccharomyces yeast is NMI accession no. V08/013,411 or NMI accession no. V09/005,064.

In another embodiment, the Saccharomyces strain is a mutant of NMI V08/013,411, or a mutant of V09/005,064. Mutants of NMI V08/013,411 or V09/005,064 may be produced by exposing diploid or haploid cells from these strains to a mutagen. The mutagen may be any mutagen suitable for mutagenising the genome of Saccharomyces yeast cells. Typical mutagens include UV light, alkylating agents, ionising radiation. Examples of alkylating agents include N-ethyl-N-nitrosourea, cyclophosphamide, N-nitroso-N-methylurea, 1-methyl-3-nitro-1-nitrosoguanidine, dimethylsulphate, ethyl methanesulphonate. Examples of ionising radiation include gamma-radiation, X-ray radiation, beta-radiation, alpha-radiation. Methods for producing mutants of Saccharomyces yeast, and specifically mutants of Saccharomyces cerevisiae, are known in the art and described in, for example, Lawrence C. W. (1991) Methods in Enzymology, 194: 273-281.

In another embodiment, the Saccharomyces strain is a derivative of NMI accession no. V08/013,411, or a derivative of NMI accession no. V09/005,064. A derivative of NMI accession nos. V08/013,411 or V09/005,064 may be a recombinant derivative or a non-recombinant derivative.

A non-recombinant derivative of NMI V08/013,411 may be produced by combining the genome of NMI V08/013,411 with the genome of a desired Saccharomyces strain to produce a Saccharomyces strain which comprises the properties of NMI V08/013,411. A non-recombinant derivative of NMI accession no. V09/005,064 may be produced by combining the genome of NMI V09/005,064 with the genome of a desired Saccharomyces strain to produce a Saccharomyces strain which comprises the properties of NMI V09/005,064. Typically, the derivative further comprises one or more properties of the desired Saccharomyces strain. Methods for combining the genome of two strains of Saccharomyces are known in the art and include, for example, mating or protoplast fusion.

Methods for mating of strains of Saccharomyces typically comprise sporulating the Saccharomyces yeast strains to be mated to form spores. The spores are germinated to form haploid Saccharomyces yeast which are then mated to produce progeny. The progeny may then be screened for desired characteristics inherited from each parent strain. Methods for mating of Saccharomyces strains are known in the art and are described in, for example, Ausubel, F. M. et al. (1997), Current Protocols in Molecular Biology, Volume 2, pages 13.2.1 to 13.2.5, published by John Wiley & Sons Inc. By way of example, sporulation of Saccharomyces yeast strains may be carried out for example as described in Ausubel, F. M. et al. (1997), Current Protocols in Molecular Biology, Volume 2, pages 13.2.1 to 13.2.5, published by John Wiley & Sons Inc. Subsequently, the spores from the yeast may be germinated by plating on to a solid medium such as GYP containing 1% w/v D-glucose, 0.5% yeast extract, 1% w/v bacteriological peptone and 1.5% w/v agar and incubated, typically at 30° C. for three to five days. Individual germinated spores usually form discrete colonies that can be isolated in pure form by streak-plating for instance on a GYP agar medium using standard microbiological techniques. The purified germinated spores can then be assayed to determine if they posses mating type “a” or “alpha”. Mating type may be determined by mixing germinated spores of Saccharomyces cerevisiae with laboratory tester strains with known mating types. Examples of laboratory tester Saccharomyces cerevisiae strains include DE6.1D and W303-1A, described for example in Attfield P. V. et al. (1994) “Concomitant appearance of intrinsic thermotolerance and storage of trehalose in Saccharomyces cerevisiae during early respiratory phase of batch-culture is CIF1-dependent”, Microbiology, 140:2625-2632. Germinated spores of mating type “a” usually flocculate, i.e. forming a granular cell suspension visible by eye, when incubated in the presence of laboratory strain DE6.1D (of known mating type “alpha”) and germinated spores of mating type “alpha” usually flocculate in the presence of laboratory strain W303-1A (of known mating type “a”). The germinated spores that show flocculation with one mating type tester strain do not show flocculation with the other tester strain. Germinated spores of mating type “alpha” generated from one strain of Saccharomyces cerevisiae may then be mated with germinated spores of mating type “a” generated from another strain, or the same strain.

Protoplast fusion typically involves the removal of cell walls from Saccharomyces yeast cells, typically using lytic enzyme, and subsequent mixing of the strains in the presence of a fusogenic agent such a polyethylene glycol, dimethyl sulphoxide and Ca²⁺ to allow fusion between protoplasts to occur. The fused cells are typically then incubated in osmotically stabilised medium to allow the cell wall of the cells to regenerate. Methods for protoplast fusion of strains of Saccharomyces are know in the art and are described in, for example, U.S. Pat. No. 4,973,560.

A recombinant derivative of NMI V08/013,411 is a strain produced by introducing into NMI V08/013,411 a nucleic acid using recombinant DNA technology. A recombinant derivative of NMI V09/005,064 is a strain produced by introducing into NMI V09/005,064 a nucleic acid using recombinant DNA technology. Methods for the introduction of nucleic acid into Saccharomyces yeast cells, and in particular strains of Saccharomyces, are known in the art and are described in, for example, Ausubel, F. M. et al. (1997), Current Protocols in Molecular Biology, Volume 2, pages 13.7.1 to 13.7.7, published by John Wiley & Sons Inc.

The Saccharomyces strain referred to above may be used in the production of ethanol from any substance which is suitable for fermentation to produce ethanol.

Yeast produced by aerobic culture of yeast on lignocellulosic hydrolysate or on substrate obtained from fermentation and distillation of hydrolysate may be used for purposes other than fermentation of lignocellulosic hydrolysates, such as: feed yeast, food yeast, yeast for other fermentations such as beer, wine, potable spirits, non-potable ethanol, leavened baked goods, source for yeast autolysates, yeast extracts and various yeast byproducts such as enzymes, vitamins, nucleotides etc.”

Test T1: Growth using xylose as sole carbon source Yeast strains are streaked onto on Glucose Yeast extract Bacteriological Peptone medium solidified with 2% Agar using standard microbiological techniques. After incubation for 72 hours at 30 deg Celsius, yeast cells are taken from plates using a sterile microbiological loop and inoculated to an OD₆₀₀ (Optical Density at 600 nm) of between 0.1 and 0.2 units (OD₆₀₀ at T₀) in 50 ml of broth. An OD₆₀₀ of 0.1 unit is equal to approximately 9×10e5 yeast cells/mL. The broth contains xylose (5% w/v), Difco Yeast Nitrogen Base w/o amino acids (0.67%), citric acid (0.3%) and trisodium citrate (0.7%) in distilled water in a 250 ml Erlenmeyer flask. Citric acid and trisodium citrate were provided as buffering agents that are not able to be used as growth substrates by Saccharomyces. D-(+)-Xylose 99% pure was obtained from Sigma-Aldrich (catalogue number X1500-500G). Cultures are incubated at 30 deg Celsius with shaking at 220 rpm (10 cm orbital diameter) for 48 hours prior to measuring OD₆₀₀ (OD at T₄₈ₕᵣₛ). The fold increase in biomass is determined by the equation:

$$\frac{{OD}_{600} at {T}_{48 hrs}}{{OD}_{600} at {T}_{0}} .$$

The invention will now be described in detail by way of reference only to the following non-limiting examples.

### EXAMPLES

### Example 1

### Growth of Saccharomyces Strain NMI V08/013,411 and Saccharomyces Strain V09/005,064 Using Xylose as a Sole Carbon Source in Minimal Mineral Medium

Saccharomyces cerevisiae strain V08/013,411 was deposited at the National Measurement Institute, 51-65 Clarke Street, South Melbourne, Victoria, 3205, Australia under the Budapest Treaty on 23 May 2008 under accession number V08/013,411.

Saccharomyces cerevisiae strain V09/005,064 was deposited at the National Measurement Institute, 1/153 Bertie Street, Port Melbourne, Victoria, 3207, Australia, under the Budapest Treaty on 18 Feb. 2009 under accession number V09/005,064.

Saccharomyces yeast strain ER, which is representative of Saccharomyces yeast used for industrial starch-to-ethanol production, was isolated using standard microbiological procedures from a pack of “Ethanol Red” dry alcohol yeast which is commercially available from Fermentis, BP 3029-137 rue Gabriel Péri, F-59703 Marcq-en-Baroeul Cedex France (batch number 470/2, production date 10/2006).

Yeast strains were grown and tested according to Test T1. Strain ER was inoculated at an optical density of 0.147 and increased to a optical density of 0.233 (1.6-fold increase) in 48 hours. Strain NMI V08/013,411 was inoculated at an optical density of 0.158 and increased to an optical density of 3.50 (22-fold increase) in 48 hours. Strain V09/005,064 was inoculated at an optical density of 0.15 and increased to an optical density of 12.20 (80-fold increase) in 48 hours.

### Example 2

### Preparation of Lignocellulosic Hydrolysates

Washed lignocellulosic biomass was dried to <5% moisture at 90-120° C. and reduced to <1.5 mm using a hammer-mill to produce dry milled biomass.

Sulphuric acid was mixed at a level of 55.2 mg sulphuric acid per gram of dry biomass, into the dry milled biomass in order to produce a 25% w/w solid/water mash.

The acidified solid/water mash was transferred to an autoclavable plastic drum, covered with aluminium foil and placed within a 100 L pressure vessel. The temperature of the pressure vessel was maintained at 130 degrees C., 23 psi/160 kPa for 90 minutes. The vessel was then depressurised to allow atmospheric pressure to be reached within 3 minutes.

The resulting hydrolyzed mash was fed into a manual screw press and dewatered. The moisture content of the pressed mash was 60-65%. Hydrolysate is liquid from the hydrolysed mash.

The hot liquor (the ‘hydrolysate’) had a pH of 0.8 to 1.5, and was continuously stirred. In order for the Saccharomyces yeast to grow in such media, a favorable pH was achieved by addition of a 2-3% v/v of 32% ammonia solution to adjust the pH of the liquor to a pH of >5 and <7 (25-30° C.). Use of ammonia provided nitrogen, which is a nutrient for Saccharomyces yeast growth and fermentation whilst simultaneously adjusting the pH of the medium into a range that is suitable for Saccharomyces yeast fermentation.

Following pH neutralization with ammonia the hydrolysate was clarified by centrifugation using a 20 cm radius swing-out rotor at 4,000 rpm for 20 min. The decanted liquor was ready to be used for Saccharomyces yeast growth and fermentation.

Hydrolysates were analysed by HPLC using a Bio-Rad Laboratories Inc. Aminex HPX-87 P column (catalogue number 125-0098) with Carbon-P Guard column (Catalogue number 125-0119). Mobile phase was HPLC grade water at a flow rate of 0.6 mL per min and a column temperature of 85 degrees Centigrade. The following table gives examples of the composition of hydrolysates achieved using the method described:

TABLE 1
Chemical compositions of hydrolysates

| | Cane trash | Corn stover |
|---|---|---|
| Glucose | 0.90 | 0.30 |
| Xylose | 2.87 | 1.87 |
| Galactose | 0.19 | 0.16 |
| Glycerol | 0.14 | 0.042 |
| Acetate | 0.86 | 0.63 |

Concentrations are given as percent weight per volume of hydrolysate liquor.

The results show that different sources of plant biomass give different values for the components analysed (and very likely for others such as phenolics, etc.) when hydrolysed by the method described. This indicates a natural degree of variability associated with plant biomass hydrolysis.

### Example 3

### High Density Inoculation of Lignocellulosic Hydrolysate Facilitates Rapid Fermentation

Saccharomyces cerevisiae strains ER and NMI V08/013,411 were grown in sucrose-minimal medium and harvested as described in Myers et al. (1997) Applied and Environmental Microbiology, 63: 145-150, to provide yeast biomass for inoculum.

One hundred milliliters of pH adjusted hydrolyzate liquor was buffered by addition of 1% tri-sodium citrate to pH 5.0. Citrate buffering was used to help maintain subsequent culture pH within a suitable range for yeast growth. pH control in culturing and fermentation could also be achieved by titration with other common acids and bases. Sixteen percent (weight per volume) D-glucose was dissolved into the hydrolyzate liquor for conversion to ethanol. The glucose was added to the hydrolyzate liquor in order to mimic the hexoses that would have been available for yeast to utilize if hydrolysed cellulose or other hexose-rich materials were employed. The buffered glucose enriched liquor was dispensed to a 250 mL conical flask.

Harvested yeast biomass was inoculated into buffered, glucose enriched cane trash hydrolyzate liquor at densities of 2×10e6, 2×10e7 and 2×10e8 yeast cells per mL of hydrolysate and incubated at 30 degrees Centigrade for 48 h. Samples were assayed for glucose and ethanol concentrations by HPLC using a Bio-Rad Laboratories Inc. Aminex HPX-87 H column (catalogue number 125-0140) with Cation-H Guard column (Catalogue number 125-0129). Mobile phase was 4 mM sulphuric acid in HPLC grade water at a flow rate of 0.6 mL per min and a column temperature of 35 degrees Centigrade. The following table shows data for ethanol produced and residual glucose following the 24 h and 48 h incubation periods.

TABLE 2
Ethanol and residual glucose in glucose enriched cane
trash hydrolysate after 24 h and 48 h fermentation
using strain NMI V08/013411 at different inocula

| Inoculum density | | 24 hour | 48 hour |
|---|---|---|---|
| 2 × 10e6/mL | Ethanol | 0.17 | 7.17 |
| | Glucose | 15.29 | 3.37 |
| 2 × 10e7/mL | Ethanol | 4.54 | 9.25 |
| | Glucose | 7.86 | 0 |
| 2 × 10e8/mL | Ethanol | 9.4 | 9.4 |
| | Glucose | 0 | 0 |

Concentrations of glucose are given as percent weight per volume, and concentrations of ethanol are given as percent volume per volume.

As shown in Table 2, the rate of conversion of glucose to ethanol by strain NMI V08/013,411 in the hydrolysate was dependent upon inoculation density. To achieve complete conversion of glucose to ethanol in 24 h required the addition of 2×10e8 cells per mL, and to achieve complete conversion of glucose to ethanol in 48 hours required the addition of 2×10e7 cells per mL. Thus using high inoculation densities of strain NMI V08/013,411 allows relatively rapid fermentations even in substrates containing inhibitors derived from the hydrolysis of lignocellulosic materials and neutralized using ammonia.

TABLE 3
Comparision of ethanol production by strains NMI V08/013411
and ER in glucose enriched cane trash hydrolysate

| Inoculum size | Strain | 24 hour | 48 hour |
|---|---|---|---|
| 2 × 10e6/mL | ER | 0.005 | 1.80 |
| | NMI | 0.17 | 7.17 |
| | V08/013411 | | |
| 2 × 10e7/mL | ER | 0.94 | 9.00 |
| | NMI | 4.54 | 9.25 |
| | V08/013411 | | |
| 2 × 10e8/mL | ER | 7.21 | 9.10 |
| | NMI | 9.40 | 9.40 |
| | V08/013411 | | |

Concentrations of ethanol are given as percent volume per volume.

As shown in Table 3, efficiency of ethanol production was dependent upon inoculum size. Strain NMI V08/013,411 exhibited more rapid and efficient fermentation than strain ER under the same conditions. In particular, strain NMI V08/013,411 was able to produce significant ethanol after 24 h when inoculated at 2×10e7/mL, and was able to achieve virtually complete theoretical fermentation of glucose to ethanol within 24 hours when inoculated at 2×10e8/mL. This data demonstrates the tolerance of strain NMI V08/013,411 to inhibitory effects of lignocellulosic hydrolysate.

### Example 4

### Fermentation of Lignocellulose Hydrolysate and Preparation of Dunder

One hundred milliliters of pH adjusted hydrolyzate liquor was buffered by addition of 1% tri-sodium citrate to pH 5.0. Citrate buffering was used to help maintain subsequent culture pH within a suitable range for yeast growth. pH control in culturing and fermentation could also be achieved by titration with other common acids and bases. Sixteen percent (weight per volume) D-glucose was dissolved into the hydrolyzate liquor for conversion to ethanol. The glucose was added to the hydrolyzate liquor in order to mimic the hexoses that would have been available for yeast to utilize if hydrolysed cellulose or other hexose-rich materials were employed. The buffered glucose enriched liquor was dispensed to a 250 mL conical flask.

Saccharomyces cerevisiae strain NMI V08/013,411 was grown in sucrose-minimal medium and harvested as described in Myers et al. (1997) Applied and Environmental Microbiology, 63: 145-150, to provide yeast biomass for inoculum.

Harvested yeast biomass was inoculated into buffered, glucose enriched hydrolyzate liquor to a density of 3.5×10e8 yeast cells per mL and incubated at 100 rpm to keep yeast suspended at 30 degrees Centigrade. Fermentation was allowed to proceed until all glucose was utilized as determined by HPLC analysis.

After the fermentation the fermented hydrolysate was centrifuged at 3,000 rpm using a rotor of 18 cm radius for 5 min. The ethanol was boiled off. Sterile distilled water was added to the remaining liquor to bring it to its pre-boiling volume. This provided dunder substrate on which to grow yeast for subsequent fermentation of freshly prepared hydrolysate liquor.

### Example 5

### Growth of Saccharomyces cerevisiae on Dunder

Foregoing examples show the advantage of having high cell densities for inoculation into hydrolysates. However, provision of high densities of pre-conditioned yeast cells for inoculation into inhibitory hydrolysates is industrially challenging because the cost of using traditional yeast inocula at such high doses is economically prohibitive. The inventors reasoned that yeast having properties such as those of NMI V08/013,411 or NMI V09/005,064 could be grown on waste products of hydrolysate fermentations (e.g. dunder), to provide high cell densities of pre-conditioned yeasts and thus make use of what is currently regarded a waste stream of hydrolysate fermentation to produce yeast biomass and yeast products.

Strains NMI V08/013,411 and ER were streaked onto GYP plates which were: 2% w/v glucose, 1% w/v bacteriological peptone, 0.5% w/v yeast extract and 2% w/v agar (GYP agar). Plates were incubated at 30 degrees Centigrade for 24 h. Yeast strains were scraped from the surface of agar plates and resuspended, separately, in 0.5 mL sterile distilled water. Cells of strain of ER were inoculated to an initial density of 2.3×10e7 per mL and cells of strain NMI V08/013,411 were inoculated to an initial density of 1.1×10e7 per mL into 100 mL freshly prepared dunder substrate from fermented cane trash hydrolysate contained in 500 mL baffled conical flasks. Flasks were incubated at 30 degrees Centigrade and 255 rpm in an orbital shaker with 10 cm orbit diameter for 4 days. Strain ER remained at 2.4×10e7 cells per mL, whereas strain NMI increased to a density of 3.4×10e8 cells per mL. These data indicate that it is possible to cultivate a strain with properties of NMI V08/013,411 using dunder as a substrate to achieve high densities of cells for subsequent uses.

### Example 6

### Growth of Saccharomyces cerevisiae on Cane Trash Dunder and Subsequent Fermentation of Cane Trash Hydrolysate

Strain NMI V08/013,411 was streaked onto GYP plates. Plates were incubated at 30 degrees Centigrade for 24 h. Yeast was scraped from the surface of agar plates and resuspended, in 0.3 mL sterile distilled water. Suspended yeast was inoculated at a density of 3.0×10e7 cells per mL into 10 mL of dunder substrate obtained from fermentation and distillation of Cane trash hydrolysate (see Example 4), contained in a 50 mL volume sterile, capped PP-test tubes (Cellstar Greiner bio-one). Tubes were incubated at 30 degrees Centigrade and 255 rpm in an orbital shaker with 10 cm orbit diameter. After 50 h incubation the 10 mL cultures were transferred separately into 50 mL of dunder from Cane trash hydrolysate contained in 250 mL baffled conical flasks, which were incubated at 30 degrees Centigrade and 255 rpm in an orbital shaker with 10 cm orbit diameter. After a further 72 h the 50 mL culture was transferred into a further 50 mL of dunder from Cane trash hydrolysate contained in 500 mL baffled conical flasks, which was incubated at 30 degrees Centigrade and 255 rpm in an orbital shaker with 10 cm orbit diameter for a further 44 h. Strain NMI V08/013,411 grew to a final cell density of 6.6×10e8 per mL under the conditions described. Yeast cells were harvested by centrifugation at 3,000 rpm using a swing-out rotor of 18 cm radius for 5 min. The liquid phase (spent dunder) from the culturing process was analysed for chemical composition and for chemical oxygen demand. The following table gives data for chemical composition of starting (fresh) dunder and final (spent) dunder.

TABLE 4
Chemical compositions of fresh dunder from Cane trash hydrolysate
and spent dunder from Cane trash following growth of yeast
strain NMI V08/013411 in the fresh dunder

| | NMI | NMI |
|---|---|---|
| | V08/013411 | V08/013411 |
| | Fresh Dunder | Spent Dunder |
| Glucose | 0 | 0 |
| Galactose | 0.10 | 0.03 |
| Xylose | 1.80 | 0.33 |
| Xylitol | 0.15 | 0 |
| Glycerol | 0.67 | 0 |
| Acetate | 0.77 | 0 |
| Ethanol | 0.29 | 0 |

Concentrations are given as percent weight per volume of either fresh dunder from Cane trash or spent dunder from Cane trash, except for ethanol which is given as percent volume per volume.

The results indicate that under the incubation conditions strain NMI V08/013,411 was able to adapt to the conditions of the dunder and utilized a wide range of residual carbon compounds, including xylose, found in the fresh dunder. The ability to utilize xylose, which is the most abundant carbon source in the dunder, enabled strain NMI V08/013,411 to grow and thereby increase its biomass markedly. Such properties of NMI V08/013,411 enable a process whereby sufficient yeast biomass can be grown on dunder to allow for subsequent inoculation of inhibitory lignocellulosic hydrolysates with sufficient yeast cells to allow efficient fermentation.

Yeast obtained via aerobic growth on Cane trash dunder substrate were used to inoculate and ferment buffered glucose enriched Cane trash hydrolysate as described in Example 4. Strain NMI V08/013,411 was inoculated into buffered glucose enriched Cane trash hydrolysate at a starting density of 5.8×10e8 cells per mL. Results of analysis of the fermentations by strain NMI V08/013,411 are shown in the following table.

TABLE 5
Analyses of fermentations of Cane trash hydrolysate
inoculated with strain NMI V08/013411 following
cultivation in dunder from Cane trash.

| | NMI | NMI |
|---|---|---|
| | V08/013411 | V08/013411 |
| | Start | 45 h |
| Glucose | 16 | 0 |
| Galactose | 0.19 | 0.10 |
| Xylose | 2.60 | 1.80 |
| Xylitol | 0 | 0.07 |
| Glycerol | 0.14 | 0.77 |
| Acetate | 0.86 | 1.11 |
| Ethanol | 0 | 8.9 |

Concentrations are given as percent weight per volume, except for ethanol which is given as percent volume per volume.

Strain NMI V08/013,411 fermented all the available glucose and produced ethanol. Strain NMI V08/013,411 shows advantageous characteristics since it will grow on dunder to produce sufficient cell mass to enable an inoculum density that will support efficient fermentation in hydrolysate.

### Example 7

### Growth of Saccharomyces cerevisiae on Corn Stover Dunder and Subsequent Fermentation of Corn Stover Hydrolysate

Corn stover hydrolysate liquor was prepared according to Example 2. Dunder substrate of Corn stover hydrolysate liquor was prepared according to Example 4. Strain NMI V08/013,411 was grown on GYP agar as described in Example 6. The yeast cells were scraped from the agar plate surface and resuspended in 0.3 mL sterile distilled water. Ten millilitres of Corn stover dunder substrate contained in a 50 mL volume sterile, capped PP-test tubes (Cellstar Greiner bio-one) was inoculated with strain NMI V08/013,411 to a density of 2.4×10e7 cells per mL. Tubes were incubated at 30 degrees Centigrade and 255 rpm in an orbital shaker with 10 cm orbit diameter. After 20 h incubation the 10 mL cultures were transferred into 90 mL of dunder from Corn stover hydrolysate contained in 500 mL baffled conical flasks, which were incubated in the same orbital shaking conditions for a further 121 h. Strain NMI grew to a final cell density of 4.2×10e8 per mL.

Yeast cells were harvested by centrifugation at 3,000 rpm using a swing-out rotor of 18 cm radius for 5 min. The liquid phase (spent dunder) from the culturing process was analysed for chemical composition and for chemical oxygen demand. The following table gives data for chemical composition of starting (fresh) dunder and final (spent) dunder.

TABLE 6
Chemical compositions of fresh dunder from Corn stover hydrolysate
and spent dunder from Corn stover following growth of yeast
strain NMI V08/013411 in the fresh dunder

| | NMI | NMI |
|---|---|---|
| | V08/013411 | V08/013411 |
| | Fresh Dunder | Spent Dunder |
| Glucose | 0 | 0 |
| Galactose | 0.14 | 0.10 |
| Xylose | 1.50 | 0.49 |
| Xylitol | 0.08 | 0.11 |
| Glycerol | 0.44 | 0.13 |
| Acetate | 0.39 | 0 |
| Ethanol | 0.28 | 0 |

Concentrations are given as percent weight per volume of either fresh dunder from Cane trash or spent dunder from Cane trash, except for ethanol which is given as percent volume per volume.

Data in Table 6 shows that strain NMI V08/013,411 was able to utilise various available carbon compounds in the dunder.

Yeast obtained via aerobic growth on Corn stover dunder substrate was used to inoculate and ferment buffered glucose enriched Corn stover hydrolysate as described in Example 4. The inoculation density was 2.8×10e8 cells per mL.

Results of analysis of the fermentation of Corn stover hydrolysate liquor strain NMI V08/013,411 is shown in the following table.

TABLE 7
Analyses of fermentation of Corn stover hydrolysate
inoculated with strain NMI V08/013411 following
cultivation in dunder from Corn stover.

| | NMI | NMI |
|---|---|---|
| | V08/013411 | V08/013411 |
| | Start | 45 h |
| Glucose | 16 | 0 |
| Galactose | 0.16 | 0.14 |
| Xylose | 1.9 | 1.5 |
| Xylitol | 0 | 0.08 |
| Glycerol | 0.042 | 0.48 |
| Acetate | 0.63 | 0.60 |
| Ethanol | 0 | 8.9 |

Concentrations are given as percent weight per volume, except for ethanol which is given as percent volume per volume.

Strain NMI V08/013,411 fermented all the available glucose and produced ethanol.

### Example 8

### Reduction of Chemical Oxygen Demand (COD) of Dunder Following Aerobic Culture of Saccharomyces cerevisiae on Dunder

Yeast strain NMI V08/013,411 was inoculated into dunders derived from fermentation of cane trash and corn stover hydrolysates and cultivated aerobically as described in Examples 6 and 7. The CODs of the danders prior to, and after the aerobic culturing process, were assayed using Spectroquant COD kits (catalogue number 1.14555.0001) supplied by Merck KGaA, 64271 Darmstadt, Germany. Kits were used according to the manufacturer's instructions. The method is USEPA approved for wastewater and is analogous to EPA 410.4, US Standard Methods 5220 D, and ISO 15705. The COD of the cane trash dunder following cultivation of strain NMI V08/013,411 in the dunder was reduced by 41%. The COD of the corn stover dunder following cultivation of strain NMI V08/013,411 in the dunder was reduced by 25%.

### Example 9

### Aerobic Propagation of Yeast on Hydrolysate to Produce Yeast Biomass

Strains ER and NMI V08/013,411 were grown on GYP plates as described in Example 5. Harvested cells were inoculated to a density of 2.3×10e7 per mL for strain ER and 1.2×10e7 per mL for strain NMI V08/013,411 in 100 mL fresh cane trash hydrolysate contained in 500 mL baffled conical flasks. The cultures were incubated at 30 degrees Centigrade and 240 rpm in an orbital shaker with 10 cm diameter orbit for 96 hours to allow growth of yeast biomass. At 96 hours, ER had not increased in cell density (1.8×10e7 per mL), but NMI V08/013,411 had increased 40-fold in cell density (4.9×10e8 per mL). As shown in Table 8 the acetate, glycerol and xylose concentrations of the hydrolysate had been reduced by Saccharomyces yeas't NMI V08/013,411.

The Saccharomyces yeast NMI V08/013,411 biomass so produced could be used for any purpose for which yeast can be used such as fermentation, food, feed, or extracts.

TABLE 8
Chemical compositions of cane trash hydrolysate prior
to and after 96 h incubation with yeast inocula.

| | |
|---|---|
| ER | NMI V08/013411 |

| | Start | 96 hr | Start | 96 hour |
|---|---|---|---|---|
| Glucose | 0.70 | 0.53 | 0.71 | 0 |
| Galactose | 0.11 | 0.11 | 0.12 | 0.06 |
| Xylose | 2.41 | 2.45 | 2.44 | 1.18 |
| Glycerol | 0.12 | 0.12 | 0.12 | 0.03 |
| Acetate | 0.79 | 0.81 | 0.80 | 0 |

Concentrations are given as percent weight per volume.

### Example 10

### Ethanol Production by Strain V09/005,064 Using Glucose or Sucrose as Free Sugars Within Cane Trash Hydrolysate Background

This example shows that strain V09/005,064 can be propagated in cane trash hydrolysate and the yeast biomass produced via that propagation can ferment free glucose or sucrose in the cane trash hydrolysate broth.

Strain V09/005,064 was grown on GYP plates (as described in Example 5). Cane trash hydrolysate was prepared as described in Example 2 and had the composition shown in Table 9. The yeast was inoculated from the GYP plate into 50 mL of cane trash hydrolysate within a 250 mL Erlenmeyer flask and incubated at 30 degrees Centigrade with shaking at 180 rpm in an orbital shaker with 10 cm orbit diameter.

TABLE 9
Analysis of fresh cane trash hydrolysate
prior to any sugar supplementation.

| | Cane Trash |
|---|---|
| | Hydrolysate |
| Glucose | 0.43 |
| Galactose | 0.18 |
| Xylose | 2.17 |
| Xylitol | 0 |
| Glycerol | 0.026 |
| Acetate | 0.70 |
| Ethanol | 0 |

Concentrations are given as a percentage weight per volume.

After 24 h the yeast suspension was centrifuged for 10 min at 3,000 rpm and room temperature in a swing out rotor of 120 cm radius. All but 4 mL of the supernatant was decanted and the V09/005,064 yeast cells were resuspended in the remaining liquid.

i) Sucrose Fermentation in Hydrolysate

Two mL of V09/005,064 yeast that had been grown on hydrolysate was inoculated into 100 mL of fresh cane trash hydrolysate supplemented with sucrose. The initial cell density was 3.7×10e7 yeast cells per mL and the fermentation was performed in a 250 mL Erlenmeyer flask. After incubating for 48 h at 37° C. with shaking at 95 rpm, the 100 mL cane trash hydrolysate supplemented with sucrose had produced 1.8×10e8 yeast cells per mL and 12.67% v/v ethanol. Table 10 shows there was no residual sucrose after 48 h. It can be seen that strain V09/005,064 is capable of rapid fermentation of sucrose in the presence of hydrolysate prepared as described in Example 2.

TABLE 10
Fermentation of Sucrose Enriched Cane Trash Hydrolysate

| | 0 h | 48 h |
|---|---|---|
| Sucrose | 17.70 | 0 |
| Glucose | 0.31 | 0.30 |
| Galactose | 0.21 | 0.25 |
| Fructose | 0.21 | 0 |
| Xylose | 2.09 | 2.85 |
| Xylitol | 0 | 0.12 |
| Glycerol | 0.023 | 0.57 |
| Acetate | 0.62 | 0.56 |
| Ethanol | 0 | 12.67 |

Concentrations are given as a percentage weight per volume, except for ethanol which is given as percent volume per volume.

ii) Glucose Fermentation in Hydrolysate

Two mL of V09/005,064 yeast that had been grown on hydrolysate was inoculated into 100 mL of fresh cane trash hydrolysate supplemented with glucose. The initial cell density was 3.8×10e7 yeast cells per mL and the fermentation was performed in a 250 mL Erlenmeyer flask. After incubating for 48 h at 37° C. with shaking at 95 rpm, the 100 mL cane trash hydrolysate supplemented with glucose had produced 1.8×10e8 yeast cells per mL and 12.61% v/v ethanol. Table 11 shows there was less than 1% w/v residual glucose after 48 h. It can be seen that strain V09/005,064 is capable of rapid fermentation of glucose in the presence of hydrolysate prepared as described in Example 2.

TABLE 11
Fermentation of Glucose Enriched Cane Trash Hydrolysate

| | 0 h | 48 h |
|---|---|---|
| Glucose | 17.61 | 0.96 |
| Galactose | 0.18 | 0.17 |
| Xylose | 1.98 | 1.81 |
| Xylitol | 0 | 0 |
| Glycerol | 0.023 | 0.53 |
| Acetate | 0.61 | 0.56 |
| Ethanol | 0 | 12.61 |

Concentrations are given as a percentage weight per volume, except for ethanol which is given as percent volume per volume.

### Example 11

### Propagation of Saccharomyces cerevisiae in a Mixture of Cane Trash Hydrolysate and Dunder and Subsequent Simultaneous Saccharification and Fermentation to Produce Ethanol

This example shows that strain V09/005,064 grows efficiently in a mixture of freshly hydrolysed sugar cane trash and dunder. The results also show that the quantity of yeast produced is sufficient to provide both yeast for a simultaneous saccharification and fermentation process and excess yeast for other useful purposes for which yeast widely used. Thus it is possible through the described process to produce ethanol and excess yeast biomass.

### Preparation of Seed Culture for Aerobic Fermentation:

To prepare a seed culture of Strain V09/005,064 for inoculation into a hydrolysate/dunder aerobic propagation, strain V09/005,064 was grown on GYP plates as described in Example 5 and inoculated into 50 mL of cane trash hydrolysate contained in a 250 mL Erlenmeyer flask. The hydrolysate, prepared as per Example 2, had the composition shown in TABLE 12. The Erlenmeyer flask was incubated at 30 degrees Centigrade and 180 rpm in an orbital shaker with 10 cm orbit diameter. After 24 h, the yeast suspension was halved into 2×25 mL samples and each was resuspended separately into 75 mL of cane trash hydrolysate. Each of the resulting 100 mL yeast in cane trash hydrolysate suspensions were placed into 500 mL baffled Erlenmeyer flasks, and incubated for a further 72 h under the same conditions as described above. After 72 h the flask cultures were combined to produce the seed culture for aerobic propagation of V09/005,064 in a dunder-hydrolysate mix.

The dunder-hydrolysate mix was prepared by blending 500 mL dunder derived from a previous round of fermentation with 300 mL fresh cane trash hydrolysate. The final composition of the blend is shown in TABLE 12. It was introduced into a 3.1 L fermentation vessel with dimensions of 21 cm height to 14 cm diameter, and the conditions for propagation of yeast in the fermentation vessel were as listed in TABLE 13. The remaining 500 mL of dunder and cane trash hydrolysate mix was continuously stirred whilst being fed into the fermentation vessel at 0.65 mL/min.

Over a 67 h period the yeast cells increased from a density of 8.7×10e7 per mL to a density of 7.8×10e8 per mL.

TABLE 12
Analyses of cane trash hydrolysate, dunder-hydrolysate
mix and spent dunder from the yeast propagation phase

| | | Dunder- | |
|---|---|---|---|
| | Cane Trash | Hydrolysate | Spent media |
| | Hydrolysate | Mix for aerobic | after aerobic |
| | for growth of | propagation | propagation |
| | seed culture | in fermenter | in fermenter |
| Glucose | 0.35 | 0.40 | 0 |
| Galactose | 0.094 | 0.37 | 0.028 |
| Xylose | 1.95 | 1.98 | 0.14 |
| Xylitol | 0 | 0.23 | 0 |
| Glycerol | 0.10 | 1.53 | 0 |
| Acetate | 0.55 | 0.74 | 0.039 |
| Ethanol | 0 | 0.16 | 0 |

Concentrations are given as a percentage weight per volume, except for ethanol which is given as percent volume per volume.

TABLE 13
Conditions for the Propagation phase on dunder-
hydrolysate mix, in 3.1 L Fermentation Vessel.

| Fermenter Parameters: | |
|---|---|
| (Propagation Phase) | Set point Conditions |
| Maximum volume in | 3.1 Litres |
| vessel | |
| Working volume | 1,000 ± 500 millilitres |
| Temperature | 30.0 ± 2 degrees |
| | centigrade |
| Aeration into vessel | 2.0 ± 0.5 Litres of |
| | sterile air per minute |
| Agitation | 600 ± 50 revolutions per |
| | minute using a single |
| | 6-paddle impeller set |
| | 30% from the base of |
| | the bottom driven |
| | stirrer shaft |
| pH control | 5.0 ± 0.5 pH units, |
| | using 2MNaOH and |
| | 1MH2SO4 as the |
| | controlling agents. |
| Foaming control | Using a direct contact |
| | probe, connected to a |
| | peristaltic pump with |
| | 0.5% w/v of Henkel's |
| | Antifoam fed when |
| | required. |

After 67 h the yeast was harvested by centrifugation using a 20 cm radius swing-out rotor at 4,000 rpm for 20 min. Harvested yield was 13.7 g of dry yeast. As shown in Table 12, the composition of the spent media after aerobic propagation in fermenter indicates that the yeast V09/005,064 had completely metabolised all the glucose, xylitol, glycerol and ethanol, and left only residual levels of galactose, xylose and acetate each of which had been reduced significantly.

Chemical oxygen demand assays were performed as described in Example 8. These assays showed there was a 46% reduction in the COD of the spent media after aerobic propagation in the fermenter when compared to the original dunder-hydrolysate mix.

### Simultaneous Saccharification and Fermentation Using V09/005,064:

Approximately one third of the harvested strain V09/005,064 cell biomass was resuspended in 1 L of fresh cane trash hydrolysate of composition shown in Table 14, to give a density of 1.7×10e8 per mL. To perform a simultaneous saccharification and fermentation, the hydrolysate containing the V09/005,064 culture was supplemented with 200 g maltodextrin, and 1 mL of Spirizyme Fuel (Novozymes Australia Pty Ltd) was injected into the vessel to effect saccharification of the maltodextrin.

TABLE 14
Composition of Hydrolysate used.

| | Cane Trash |
|---|---|
| | Hydrolysate |
| Glucose | 0.44 |
| Galactose | 0.11 |
| Xylose | 2.73 |
| Xylitol | 0 |
| Glycerol | 0.019 |
| Acetate | 0.79 |
| Ethanol | 0 |

Concentrations are given as a percentage weight per volume, except for ethanol which is given as percent volume per volume.

Fermentation conditions were as shown in Table 15.

TABLE 15
Conditions for the Simultaneous Saccharification and
fermentation phase in cane trash hydrolysate supplemented
with maltodextrin and Spirizyme ® Fuel (Novozymes)

| Fermenter Parameters | |
|---|---|
| (Propagation Phase) | Set point Conditions |
| Maximum volume in | 3.1 Litres |
| vessel | |
| Working volume | 1,000 ± 500 millilitres |
| Temperature | 30.0 ± 2 degrees |
| | centigrade |
| Aeration into vessel | None |
| Agitation | 200 ± 50 revolutions per |
| | minute using a single |
| | 6-paddle impeller set |
| | 30% from the base of |
| | the bottom driven |
| | stirrer shaft |
| pH control | 5.0 ± 0.5 pH units, |
| | using 2MNaOH and |
| | 1MH2SO4 as the |
| | controlling agents. |
| Foaming control | Using a direct contact |
| | probe, connected to a |
| | peristaltic pump with |
| | 0.5% w/v of Henkel's |
| | Antifoam fed when |
| | required. |

As shown in Table 16, the V09/005,064 yeast propagated in hydrolysate-dunder produced 8.39% ethanol within 46 h at 30 degrees Centigrade in the simultaneous saccharification and fermentation. This fermentation was performed in the presence of hydrolysate produced as described in Example 2.

TABLE 16
Simultaneous saccharification using maltodextrin and
Spirizyme ® Fuel enzyme, and fermentation
by strain V09/005064 in a cane trash hydrolysate background.

| | 0 h | 46 h |
|---|---|---|
| Glucose | 0 | 2.49 |
| Galactose | 0.15 | 0.13 |
| Xylose | 2.41 | 2.23 |
| Xylitol | 0 | 0 |
| Glycerol | 0.031 | 0.75 |
| Acetate | 0.67 | 0.76 |
| Ethanol | 0.018 | 8.49 |

Concentrations are given as a percentage weight per volume, except for ethanol which is given as percent volume per volume.

Since only ⅓ of the V09/005,064 yeast was required for an effective simultaneous saccharification and fermentation, it is clear that the excess yeast biomass could be used for any one or multiple purposes as whole cells or extracted cells. Such purposes would be known to those skilled in the art, and include use as feed, to produce vitamins, nucleotides, yeast extract, etc.

### Example 12

### Propagation of Saccharomyces cerevisiae in Cane Trash Hydrolysate and Subsequent Simultaneous Saccharification and Fermentation of Cellulosic Material to Produce Ethanol

This example demonstrates the ability of the strain V09/005,064 to perform a simultaneous saccharification and fermentation of cellulose in the presence of hydrolysate prepared as described in Example 2. It also demonstrates the industrial utility of generating large quantities of yeast from the hydrolysate since it is shown that a low inoculum of yeast is inhibited by the hydrolysate, whereas a high inoculum of yeast effectively detoxifies the hydrolysate and results in a more efficient simultaneous saccharification and fermentation.

Cane trash hydrolysate was prepared as described in Example 2. The composition of cane trash hydrolysate is shown in Table 17:

TABLE 17
Composition of Hydrolsyates used.

| | Cane Trash | Cane Trash |
|---|---|---|
| | Hydrolysate A | Hydrolysate E |
| Glucose | 0.43 | 0.43 |
| Galactose | 0.24 | 0.26 |
| Xylose | 2.11 | 2.30 |
| Xylitol | 0 | 0 |
| Glycerol | 0.026 | 0.030 |
| Acetate | 0.70 | 0.72 |
| Ethanol | 0 | 0 |

Concentrations are given as a percentage weight per volume.

Strain V09/005,064 was propagated in one litre of hydrolysate A in a 3.1 L aerated fermenter under conditions described Table 13. Strain V09/005,064 was inoculated at 1.35×10e8 cells per mL and reached a density of 6.0×10e8 cells per mL prior to harvesting. Yeast was harvested by centrifugation using a 20 cm radius swing-out rotor at 4,000 rpm for 20 min. Supernatant was decanted and the cells resuspended in the remaining spent hydrolysate to give a concentrated yeast cell suspension of 27% w/v total solids.

Avicel (Sigma Aldrich—Fluka), used as a crystalline cellulose substitute, was added to hydrolysate B (Table 17) to make 20% w/v cellulosic slurry. Cellulolytic enzymes (obtained from Novozymes Australia Pty Ltd) were added to this slurry. The enzymes were Cellulclast 1.5 L (product number CCN03100, added at 0.128 mL per gram of Avicel) and Novozym 188 (product number DCN00206, added at 0.064 mL per gram of Avicel). The enzyme-loaded cellulose slurry was split into two 25 mL lots contained in 50 mL PP-test tubes (Greiner Bio-One). Yeast strain V09/005,064 was inoculated from the 27% w/v suspension described above, into one of the enzyme-loaded slurries at 3.6×10e7 cells per mL and into the other of the enzyme-loaded slurries at 9×10e8 cells per mL. Tubes were incubated at 37 degrees Centigrade and 90 rpm. Samples from each were analysed after 24 and 48 h. Data are given in Table 18.

TABLE 18
Simultaneous saccharification using crystalline cellulose (Avicel)
and Celluclast 1.5 L and Novozym 188 enzymes, and fermentation
by strain V09/005064 in a cane trash hydrolysate background.

| | |
|---|---|
| Cellulose-laden | Cellulose-laden |
| hydrolysate | hydrolysate |
| inoculated at | inoculated at |
| 3.60 × 10e7 | 9.00 × 10e8 |
| cells of strain | cells of strain |
| V09/005064 per mL | V09/005064 per mL |

| | 24 h | 48 h | 24 h | 48 h |
|---|---|---|---|---|
| Glucose | 2.30 | 2.63 | 0 | 0 |
| Galactose | 0.26 | 0.26 | 0.22 | 0.22 |
| Xylose | 2.31 | 2.35 | 2.01 | 2.00 |
| Xylitol | 0 | 0 | 0.52 | 0.77 |
| Glycerol | 0.047 | 0.055 | 0.22 | 0.29 |
| Acetate | 0.87 | 0.89 | 0.89 | 0.93 |
| Ethanol | 0.29 | 0.44 | 2.27 | 3.06 |

Concentrations are given as a percentage weight per volume, except for ethanol which is given as percent volume per volume.

The data show that at the lower density of yeast cells ethanol production did occur but also that glucose accumulated in the simultaneous saccharification and fermentation. However, when the high cell density was inoculated, glucose did not accumulate and ethanol concentration was greater.

The results of this example show it is possible to grow yeast strain V09/005,064 in cane trash hydrolysate and to subsequently use this yeast in simultaneous saccharification and fermentation of cellulose, within a hydrolysate background.

The advantage of being able to grow yeast in hydrolysate and so produce sufficient yeast for high density inoculation into subsequent hydrolysate-cellulose fermentations is also demonstrated by this example.
In the claims which follow and in the preceding description of the invention, except where the context requires otherwise due to express language or necessary implication, the word “comprise” or variations such as “comprises” or “comprising” is used in an inclusive sense, i.e. to specify the presence of the stated features but not to preclude the presence or addition of further features in various embodiments of the invention.
It is to be understood that a reference herein to a prior art document does not constitute an admission that the document forms part of the common general knowledge in the art in Australia or any other country.

1.-61. (canceled)

62. A method of producing Saccharomyces yeast biomass or a product of a Saccharomyces yeast, comprising incubating a substrate comprising a C5 compound-containing material with a Saccharomyces yeast in conditions which cause growth of the Saccharomyces yeast or production of the product, wherein the C5 compound-containing material is:
(a) C5 compound-containing material obtained from lignocellulosic hydrolysate;
(b) C5 compound-containing material obtained from fermentation of lignocellulosic hydrolysate; or
(c) a mixture of (a) and (b),
and wherein the Saccharomyces yeast is capable of utilizing xylose as a carbon source for growth on the substrate.

63. The method of claim 62, wherein the C5 compound-containing material is obtained from lignocellulosic hydrolysate.

64. The method of claim 62, wherein the C5 compound-containing material is obtained from fermentation of lignocellulosic hydrolysate.

65. The method of claim 62, wherein the C5 compound-containing material is a mixture of C5 compound-containing material obtained from lignocellulosic hydrolysate and C5 compound-containing material obtained from fermentation of lignocellulosic hydrolysate.

66. The method of claim 62, wherein the C5 compound-containing material is xylose-containing material.

67. The method of claim 62, wherein the C5 compound-containing material is dunder.

68. The method of claim 62, wherein the C5 compound-containing material is lignocellulosic hydrolysate.

69. The method of claim 62, wherein the substrate is the C5 compound-containing material.

70. The method of claim 62, wherein the Saccharomyces yeast is grown aerobically on the substrate.

71. The method of claim 62, wherein the Saccharomyces yeast is non-recombinant.

72. The method of claim 62, wherein the Saccharomyces yeast is selected from the group consisting of: NMI Accession No. V08/013,411 or a mutant or derivative thereof having the defining properties of V08/013,411, and NMI Accession No. V09/005,064, or a mutant or derivative thereof having the defining properties of V09/005,064.

73. A method of producing ethanol, comprising the steps of:
(a) incubating Saccharomyces yeast produced by the method of claim 1, and a pH adjusted lignocellulosic hydrolysate under conditions which cause fermentation of the lignocellulosic hydrolysate to produce ethanol and C5 compound-containing material; and
(b) isolating the ethanol.

74. The method of claim 73, wherein the pH adjusted lignocellulosis hydrolysate comprises a lignocellulosic hydrolysate and an alkaline agent.

75. The method of claim 74, wherein the alkaline agent is ammonia.

76. The method of claim 73, comprising the further step of:
(c) growing Saccharomyces yeast on substrate comprising:
(i) the C5 compound-containing material produced in step (a);
(ii) C5 compound-containing material obtained from lignocellulosic hydrolysate; or
(iii) a mixture of (i) and (ii),
wherein the Saccharomyces yeast is capable of utilizing xylose as a carbon source for growth on the substrate.

77. The method of claim 76, comprising the further step of:
(d) repeating steps (a) to (c) using the Saccharomyces yeast grown in step (c).

78. The method of claim 76, wherein the substrate is the C5 compound-containing material.

79. The method of claim 76, wherein the Saccharomyces yeast is grown aerobically on the substrate.

80. The method of claim 76, wherein the Saccharomyces yeast is non-recombinant.

81. The method of claim 76, wherein the Saccharomyces yeast is selected from the group consisting of: NMI Accession No. V08/013,411 or a mutant or derivative thereof having the defining properties of V08/013,411, and NMI Accession No. V09/005,064, or a mutant or derivative thereof having the defining properties of V09/005,064.

82. A method of producing ethanol and Saccharomyces yeast biomass, comprising the steps of:
(a) incubating a pH adjusted lignocellulosic hydrolysate, and a Saccharomyces yeast capable of utilizing xylose as a carbon source for growth on a substrate obtained from fermentation of lignocellulosic hydrolysate, under conditions which cause fermentation of the lignocellulose hydrolysate to produce ethanol and a C5 compound-containing material;
(b) isolating the ethanol; and
(c) growing Saccharomyces yeast on substrate comprising:
(i) the C5 compound-containing material produced in step (a);
(ii) C5 compound-containing material obtained from lignocellulosic hydrolysate; or
(iii) a mixture of (i) and (ii),
wherein the Saccharomyces yeast is capable of utilizing xylose as a carbon source for growth on the substrate.

83. The method of claim 82, further comprising the step of:
(d) repeating steps (a) to (c) using the Saccharomyces yeast grown in step (c).

84. The method of claim 82, wherein the pH adjusted lignocellulosis hydrolysate comprises a lignocellulosic hydrolysate and an alkaline agent.

85. The method of claim 82, wherein the alkaline agent is ammonia.

86. The method of claim 82, wherein the Saccharomyces yeast are grown aerobically on the substrate.

87. The method of claim 82, wherein the Saccharomyces yeast is non-recombinant.

88. The method of claim 82, wherein the Saccharomyces yeast is selected from the group consisting of: NMI Accession No. V08/013,411 or a mutant or derivative thereof having the defining properties of V08/013,411, and NMI Accession No. V09/005,064, or a mutant or derivative thereof having the defining properties of V09/005,064.

89. A method of producing ethanol, the method comprising the steps of:
(a) providing Saccharomyces yeast capable of utilizing xylose as a carbon source for growth on a substrate obtained from fermentation of lignocellulosic hydrolysate, and a lignocellulosic hydrolysate;
(b) pH adjusting the lignocellulosic hydrolysate for fermentation by the Saccharomyces yeast, the pH adjusting comprising adjusting the pH of the hydrolysate to a pH which supports fermentation by the Saccharomyces yeast by addition of ammonia;
(c) incubating the Saccharomyces yeast and pH adjusted lignocellulosic hydrolysate in conditions which cause fermentation of the lignocellulose hydrolysate to produce ethanol and C5 compound containing material; and
(d) isolating the ethanol.

90. The method of claim 89, comprising the further step of:
(e) growing Saccharomyces yeast on substrate comprising:
(i) the C5 compound-containing material produced in step (a);
(ii) C5 compound-containing material obtained from lignocellulosic hydrolysate; or
(iii) a mixture of (i) and (ii),
wherein the Saccharomyces yeast is capable of utilizing xylose as a carbon source for growth on the substrate.

91. The method of claim 90, comprising the further step of:
(f) repeating steps (a) to (e) using the Saccharomyces yeast grown in step (e).

92. The method of claim 90, wherein the substrate is the C5 compound-containing material.

93. The method of claim 90, wherein the Saccharomyces yeast is grown aerobically on the substrate.

94. The method of claim 90, wherein the Saccharomyces yeast is non-recombinant.

95. The method of claim 90, wherein the Saccharomyces yeast is selected from the group consisting of: NMI Accession No. V08/013,411 or a mutant or derivative thereof having the defining properties of V08/013,411, and NMI Accession No. V09/005,064, or a mutant or derivative thereof having the defining properties of V09/005,064.

96. A method of producing ethanol and Saccharomyces yeast biomass, the method comprising the steps of:
(a) providing Saccharomyces yeast capable of utilizing xylose as a carbon source for growth on a substrate obtained from fermentation of lignocellulosic hydrolysate, and a lignocellulosic hydrolysate;
(b) pH adjusting the lignocellulosic hydrolysate for fermentation by the Saccharomyces yeast, the pH adjusting comprising adjusting the pH of the hydrolysate to a pH which supports fermentation by the Saccharomyces yeast by addition of ammonia;
(c) incubating the Saccharomyces yeast and pH adjusted lignocellulosic hydrolysate in conditions which cause fermentation of the lignocellulosic hydrolysate to produce ethanol and C5 compound-containing material;
(d) isolating the ethanol; and
(e) growing Saccharomyces yeast on substrate comprising:
(i) the C5 compound-containing material obtained from step (c);
(ii) C5 compound-containing material obtained from lignocellulosic hydrolysate; or
(iii) a mixture of (i) and (ii),
wherein the Saccharomyces yeast is capable of utilizing xylose as a carbon source for growth on the substrate.

97. The method of claim 96, comprising the further step of:
(f) repeating steps (a) to (e) using the Saccharomyces yeast grown in step (e).

98. The method of claim 96, wherein the Saccharomyces yeast is non-recombinant.

99. The method of claim 96, wherein the Saccharomyces yeast is selected from the group consisting of: NMI Accession No. V08/013,411 or a mutant or derivative thereof having the defining properties of V08/013,411, and NMI Accession No. V09/005,064, or a mutant or derivative thereof having the defining properties of V09/005,064.

100. A Saccharomyces yeast selected from the group consisting of: a strain having NMI Accession No. V08/013,411, or a mutant or derivative thereof which has the defining properties of NMI Accession No. V08/013,411, and a strain having NMI Accession No. V09/005,064, or a mutant or derivative thereof which has the defining properties of NMI Accession No. V09/005,064.

101. The Saccharomyces yeast of claim 100, wherein the yeast is a strain having NMI Accession No. V09/005,064, or a mutant or derivative thereof which has the defining properties of NMI Accession No. V09/005,064.

102. The Saccharomyces yeast of claim 100, wherein the yeast is NMI Accession No. V09/005,064.

103. A method of producing ethanol, the method comprising the steps of:
(a) incubating a Saccharomyces yeast selected from the group consisting of: a strain having NMI Accession No. V08/013,411, or a mutant or derivative thereof which has the defining properties of NMI Accession No. V08/013,411, and a strain having NMI Accession No. V09/005,064, or a mutant or derivative thereof which has the defining properties of NMI Accession No. V09/005,064, and a pH adjusted lignocellulosic hydrolysate under conditions which cause fermentation of the lignocellulosic hydrolysate to produce ethanol and a C5 compound-containing material; and
(b) isolating the ethanol.

104. The method of claim 103, wherein the pH adjusted lignocellulosis hydrolysate comprises a lignocellulosic hydrolysate and an alkaline agent.

105. The method of claim 103, wherein the alkaline agent is ammonia.

106. The method of claim 103, comprising the further step of:
(c) growing a Saccharomyces yeast on substrate comprising:
(i) the C5 compound-containing material produced in step (a);
(ii) C5 compound-containing material obtained from lignocellulosic hydrolysate; or
(iii) a mixture of (i) and (ii),
wherein the Saccharomyces yeast is selected from the group consisting of: a strain having NMI Accession No. V08/013,411, or a mutant or derivative thereof which has the defining properties of NMI Accession No. V08/013,411, and a strain having NMI Accession No. V09/005,064, or a mutant or derivative thereof which has the defining properties of NMI Accession No. V09/005,064.

107. The method of claim 106, comprising the further step of:
(d) repeating steps (a) to (b) using the Saccharomyces yeast grown in step (c).

108. The method of claim 106, wherein the Saccharomyces yeast are grown aerobically on the substrate.

109. The method of claim 106, wherein the substrate is the C5 compound-containing material.

110. A method of producing ethanol and Saccharomyces yeast biomass, the method comprising the steps of:
(a) incubating a Saccharomyces yeast selected from the group consisting of: a strain having NMI Accession No. V08/013,411, or a mutant or derivative thereof which has the defining properties of NMI Accession No. V08/013,411, and a strain having NMI Accession No. V09/005,064, or a mutant or derivative thereof which has the defining properties of NMI Accession No. V09/005,064, and a pH adjusted lignocellulosic hydrolysate under conditions which cause fermentation of the lignocellulosic hydrolysate to produce ethanol and a C5 compound-containing material;
(b) isolating the ethanol; and
(c) growing a Saccharomyces yeast on substrate comprising:
(i) the C5 compound-containing material produced in step (b);
(ii) C5 compound-containing material obtained from lignocellulosic hydrolysate; or
(iii) a mixture of (i) and (ii),
wherein the Saccharomyces yeast is selected from the group consisting of: a strain having NMI Accession No. V08/013,411, or a mutant or derivative thereof which has the defining properties of NMI Accession No. V08/013,411, and a strain having NMI Accession No. V09/005,064, or a mutant or derivative thereof which has the defining properties of NMI Accession No. V09/005,064.

111. The method of claim 110, further comprising the step of:
(d) repeating steps (a) to (c) using the Saccharomyces yeast grown in step (c).

112. The method of claim 110, wherein the pH adjusted lignocellulosis hydrolysate comprises a lignocellulosic hydrolysate and an alkaline agent.

113. The method of claim 112, wherein the alkaline agent is ammonia.

114. The method of claim 110, wherein the Saccharomyces yeast are grown aerobically on the substrate.

115. The method of claim 110, wherein the substrate is the C5 compound-containing material.

116. The method of claim 110, wherein the C5 compound containing material is dunder.

117. A method of producing Saccharomyces yeast biomass or a product of a Saccharomyces yeast, the method comprising incubating a substrate comprising C5 compound-containing material with Saccharomyces yeast selected from the group consisting of: a strain having NMI Accession No. V08/013,411, or a mutant or derivative thereof which has the defining properties of NMI Accession No. V08/013,411, and a strain having NMI Accession No. V09/005,064, or a mutant or derivative thereof which has the defining properties of NMI Accession No. V09/005,064, under conditions which cause growth of the Saccharomyces yeast or production of the product, wherein the C5 compound-containing material is:
(a) C5 compound-containing material obtained from lignocellulosic hydrolysate;
(b) C5 compound-containing material obtained from fermentation of lignocellulosic hydrolysate; or
(c) a mixture of (a) and (b).

118. The method of claim 117, wherein the C5 compound-containing material is dunder, lignocellulosic hydrolysate or a mixture of dunder and lignocellulosic hydrolysate.

119. The method of claim 117, wherein the yeast is grown on the substrate.

120. The method of claim 117, wherein the yeast is grown aerobically on the substrate.
